(19) United States
(12) Patent Application Publication
(10) Pub. No.: US 2007/0219605 A1
(43) Pub. Date: Sep. 20, 2007

(54) Treatment of tissue volume with radiant energy

(75) Inventors: Ilya Yaroslavsky, North Andover, MA (US); Michael Smotrich, Andover, MA (US); Gregory B. Altshuler, Lincoln, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 11/588,599

(22) Filed: Oct. 27, 2006

Related U.S. Application Data
(60) Provisional application No. 60/783,878, filed on Mar. 20, 2006.

(51) Int. Cl. A61F 2/00

(52) U.S. Cl. 607/100; 607/88

(57) ABSTRACT

Devices and methods for utilizing electromagnetic radiation and other forms of energy to treat a volume of tissue at depth are described. In one aspect, a device modulates the flux incident on surface tissue to control and vary the depth in the tissue at which an effective dose of radiant energy is delivered and, thereby, treat a specific volume of tissue. The methods and devices disclosed are used to perform various treatments, including treatments to relieve pain and promote healing of tissue.

### REFERENCE TO RELATED APPLICATION

This application claims priority to U.S. Provisional Application No. 60/783,878, Treatment of Tissue Volume With Radiant Energy, filed Mar. 20, 2006.

### BACKGROUND OF THE INVENTION

1. Technical Field

This invention relates generally to methods and devices for utilizing radiant energy, e.g., light, infrared, and other electromagnetic radiation, to treat a tissue volume located at a given depth below the tissue surface. In particular, embodiments are disclosed for treating such tissue volumes to reduce and relieve pain, to prevent and reduce fibrosis and scar formation, and to promote healing of damaged tissue.

2. Background Art

Electromagnetic radiation (“EMR”), especially visible light and infrared radiation, has been used for a number of therapeutic purposes, including as a means to reduce and relieve pain, to promote healing and to treat other clinical conditions through photobiostimulation and photobiomodulation procedures. Such treatments using EMR are referred to by various names, including, among others, Thermally Enhanced Photobiomodulation, Thermally Enhanced Photobiostimulation, Thermally Enhanced Pain Treatment (“TEPT”), Low Level Light Therapy (“LLLT”), and Low Intensity Light Therapy (“LILT”). Such treatments generally have been directed to stimulating or modulating cellular processes using visible light and/or infrared radiation (i.e., heat).

For example, low-power emitting light sources, including lasers emitting typically less than 100 mW, have been used worldwide over the past three decades to treat a variety of clinical conditions. Light has been reported to stimulate DNA synthesis, activate enzyme-substrate complexes, transform prostaglandins and produce microcirculatory effects. Several works report such effects resulting from irradiating endogenous chromophores (i.e., without application of exogenous photosensitizers) in cells or tissues.

The use of LLLT and LILT (which are essentially synonymous terms) to achieve photochemical responses is commonly referred to as photobiostimulation, photobiomodulation and photodynamic therapy. Depending on the context, these photochemical responses can involve exogenous or endogenous substances or a combination of both. In addition to laser light, photobiostimulation can be achieved using other monochromatic or quasi-monochromatic light sources (e.g., LEDs) or by suitably filtering broadband light sources (e.g., filtering fluorescent lamps, halogen lamps, incandescent lamps, discharge lamps, multi-band and broadband LEDs and natural sunlight). Biostimulation achieved by laser sources is also referred to as low-level laser therapy.

The primary mechanism of low-intensity laser/light therapy is thought to be photochemical and photobiological. The photochemical process resulting from photobiostimulation is believed to involve the integration of photons into the cellular machinery of biochemical reactions. Generally, the principle of light absorption and integration of the photon energy into the cellular respiratory cycle is a well-known natural phenomenon. Photosynthesis and vision are two examples of this phenomenon. In these processes, the photoacceptor molecules are chlorophyll and rodopsin, respectively.

In the case of photobiostimulation, several concurrent mechanisms of action have been demonstrated in vitro. One example of such a mechanism involves cytochrome c oxidase, which is a primary cellular photoacceptor of low level light. Cytochrome c oxidase is a respiratory chain enzyme residing within the cellular mitochondria, and is the terminal enzyme in the respiratory chain of eukaryotic cells. In particular, cytochrome c oxidase mediates the transfer of electrons from cytochrome c to molecular oxygen. The involvement of cytochrome c is known to be central to the redox chemistry leading to generation of free energy that is then converted into an electrochemical potential across the inner membrane of the mitochondrion, and ultimately drives the production of adenosine triphosphate (ATP). Accordingly, it has been postulated that photobiostimulation has the potential of increasing the energy available for metabolic activity of cells. The primary cellular photoacceptors of low level laser light at a range of wavelengths have been identified, for example, in “Lasers in Medicine and Dentistry,” Eds. Z. Simunovic, Vitgraf:Rijeka, 2000, pp. 97-125.

Activation of cytochrome c with light can trigger a variety of biochemical reactions leading to a range of responses at cellular, tissue, organ, and body levels. Various embodiments of LILT apparatus and techniques are known in the art. For example, such devices and techniques are described in U.S. Pat. No. 6,471,716 entitled “Low level light therapy method and apparatus with improved wavelength, temperature and voltage control” (J. P. Pecukonis).

It has been further demonstrated that photobiostimulation can be used to enhance cellular proliferation to achieve therapeutic effects. ATP molecules serve as a substrate to cyclic AMP (cAMP) which, in conjunction with calcium ions (Ca²⁺) stimulate the synthesis of DNA and RNA. cAMP is a pivotal secondary messenger affecting a plethora of physiological processes such as signal transduction, gene expression, blood coagulation and muscle contraction. Accordingly, it has been postulated that an increase in ATP production by photobiostimulation can provide a means to increase cell proliferation and protein production.

Light-stimulated ATP synthesis, such as that caused by photobiostimulation, is wavelength dependent. It has been demonstrated in vitro that prokaryotic and eukaryotic cells are sensitive to two spectral ranges, one at 350-450 nm and another at 600-830 nm. (T. I. Karu and S. F. Kolyakov, “Exact Action Spectra for Cellular Responses Relevant to Phototherapy”, Photomedicine Laser Surg. 2005, v. 23, pp. 355-361.) Karu et al. stated that the light receptors of the red wavelengths are the semichinon type of the flavoproteins of the reductase (dehydrogenases) and the cytochrome a/a3 of cytochrome c. Cytochrome c oxidase in its oxidation form is the specific chromophore of 800 through 830 nm wavelength range.

In published studies, photobiostimulation and photobiomodulation typically has been performed using relatively inexpensive sources, such as diode lasers or LEDs such as Ga—As and Ga—Al—As (e.g., emitting in the infrared spectrum (600-980 nm). Existing sources of low power laser light and light emitting diodes (LEDs) deliver powers ranging from 1 to 100 milliwatts; accordingly power densities necessary to perform photobiostimulative and photobiomodulative procedures are achieved by concentrating the light beam output into a very small spot sizes (typically less than 10 mm). This results in a typical power density at the skin surface in a range between 1 and 100 mW/cm². The small beam size makes a scanning device necessary to treat large areas. Treatment times used in most studies were in the range of 5 to 30 min. Multiple treatments are required in a majority of cases. Treatment sources and operating conditions used in conventional photobiostimulation and photobiomodulation provide negligible heating of treated tissue (e.g., less than 1° C. above normal body temperature).

The application of a thermal temperature gradient, either in the form of heat or cold, is also known in the art. In the case of heat, the ability of hyperthermia to mitigate pain has been widely used. Moreover, heat has been used in combination with low-level light therapy applied to the tissue being treated. See, e.g., U.S. Pat. No. 5,358,503 entitled “Photo-thermal therapeutic device and method” (D. E. Bertwell, J. P. Markham) (the “'503 patent”). However, such teachings generally are limited to a combination of an array of light-emitting diodes and conductive heating means. In those cases, the penetration of heat into tissue is limited to relatively shallow depths.

The use of EMR to treat pain and promote healing has been the subject of numerous studies and experiments. The scientific literature in the field has also focused on the benefits of EMR in treating inflammatory conditions, chronic joint disorders, and other conditions, such as arthritis, bursitis, carpal tunnel syndrome, fibromyalgia, hyperalgesia, lateral epicondylitis, temporomandibular joint (TMJ) dysfunction, and tendonitis. The effect of EMR on fibroblasts has been studied. The benefits of EMR in promoting healing and repair of tissue and also wound care generally, such as various types of ulcers (including diabetic ulcers, venous ulcers, and mouth ulcers), fractures, tendon damage, ligament damage, and cartilage damage has been studied. And, the effect of EMR on reducing and relieving pain, such as joint pain, lower back pain, neck pain, and pain from inflammatory conditions, has been studied.

The FDA has approved the use of EMR for the treatment of pain in certain applications, including pain associated with the head and neck and Carpal Tunnel Syndrome. While the above mechanisms have been demonstrated in numerous in vitro experiments, results of clinical trials have been so far inconclusive. Some groups have reported varying degree of success in treatment of a range of conditions. Others have observed no or minimal effect.

### SUMMARY OF THE INVENTION

One aspect of the invention is a device for treating a volume of tissue that can have: a source of EMR configured to transmit EMR to a tissue surface; and a controller electrically connected to the EMR source and configured to provide at least one control signal to the EMR source. The EMR source can be configured to emit a first level of flux and to emit a second level of flux in response to the at least one control signal, the first and second levels of flux corresponding to first and second depths below the surface of the tissue.

Preferred embodiments of this aspect of the invention can include some of the following additional features. The controller can include a modulator in electrical communication with the EMR source to control the first and second levels of flux. A cooling surface can be used for contacting the tissue surface. The cooling surface can be configured to cool the tissue when in contact with the tissue surface during operation of the device. A window can be configured to pass EMR, and can include a cooling surface for contacting the tissue surface. In some embodiments, the window can be relatively large, for example, the window can have a radiation-passing area or approximately 49 cm², and, if round, can have a diameter of approximately 7 cm. The window can be smaller for some applications, and can be even larger for other applications. For example, the optical window can comprise an area ranging from about 1 cm² to about 200 cm², about 5 cm² to about 150 cm², about 10 cm² to about 100 cm², about 25 cm² to about 75 cm², or about 30 cm² to about 60 cm² and the diameter can range from about 1 cm to about 14 cm, 2 cm to about 10 cm, or 3 cm to about 8 cm. The window or aperture can also be variable in size.

The device can be a handheld device and can also be a consumer product.

The device can include a feedback sensor configured to provide a feedback signal during operation, and a controller can be electrically connected to the feedback sensor mechanism to issue the control signals based on the information obtained from the feedback sensor. The feedback sensor can be a temperature sensor, and can be configured to measure the temperature of the tissue being treated during operation. The feedback sensor can be an optical Doppler sensor configured to measure the flow of blood within the tissue being treated.

The EMR source can be configured to provide an input flux between approximately 0.1 and 10 watts/cm². The system power can be sized to produce sufficient power for larger diameter windows, and can be relatively large for use with larger windows. For example, the system power can be on the order of 40-80 Watts and can be even larger depending on the relative size of the radiation-passing opening, such as a window or aperture. The system power can be sized to provide relatively high levels of input flux using relatively larger beam diameters and/or beam cross-sectional areas.

The device can be sized to provide sufficient input flux to allow at least a minimally effective dose of EMR to penetrate to desired tissue depths, for example, up to approximately 10 mm, 20 mm, 50 mm or more depending on the application. The term “minimally effective dose,” as used herein, refers to the lowest input flux that can penetrate to a desired tissue depth. The term “tissue depth,” as used herein, refers to how deep the radiation penetrates into the tissue.

The device can include a memory device and a processor. The device can also include input sensors, and the controller can derive treatment parameters using input data from the input sensors. The device can include one or more feedback sensors in electrical communication with the controller, and the controller can compute treatment parameters based on the sensor data. The device can utilize a lookup table containing information regarding treatment parameters.

The device can modulate the irradiance of EMR emitted from the source using intermittent pulses. The device source can include optical elements configured to provide an adjustable area of EMR that is incident on a surface of the tissue.

The device can be configured to emit a third level of input flux in response to control signals. The third level of flux can correspond to a third depth below the surface of the tissue, which can be between the first two depths or can be another depth.

Another aspect of the invention is device for treating tissue that can have a source for generating EMR, an optical window for contacting a surface of the tissue to be treated and for transmitting EMR from the source to the tissue, a cooling system in thermal communication with the optical window, the cooling system configured to remove heat from the optical window, and a modulator electrically connected to the EMR source for varying a radiant flux emitted by the EMR source from a first value corresponding to a first tissue depth to a second value corresponding to a second tissue depth.

Preferred embodiments of this aspect of the invention can include some of the following additional features. The optical window can be composed of various suitable materials such as sapphire. The device can be a handheld device or a consumer product.

The device can include one or more feedback sensors, which can be in electrical communication with the modulator. The modulator can be configured to receive a feedback signal during operation and vary the flux emitted by the EMR source in response. The feedback sensor can be of various kinds, such as a temperature sensor configured to measure the temperature of the tissue being treated or an optical Doppler sensor configured to measure the flow of blood within the tissue being treated.

The EMR source can be configured to provide an input flux in suitable ranges, such as, for example, between approximately 0.1 and 10 watts/cm2. The device can be sized to provide sufficient input flux to allow at least a minimally effective dose of EMR to penetrate to desired tissue depths, for example, up to approximately 10 mm, 20 mm, 50 mm or more depending on the application.

The device can include a memory device and a processor, for example, as part of the modulator. The device can include one or more sensors in electrical communication with the modulator. The modulator can be configured to compute treatment parameters using signals from each of the at least one sensor. The modulator can include a lookup table containing information regarding treatment parameters. The modulator can be configured to modulate the irradiance of EMR emitted from the source using intermittent pulses.

The optical window can comprise an area ranging from about 1 cm² to about 200 cm², about 5 cm² to about 150 cm², about 10 cm² to about 100 cm², about 25 cm² to about 75 cm², or about 30 cm² to about 60 cm². In some embodiments, the optical window can comprise an area greater than approximately 49 cm². The EMR source can include optical elements configured to provide an adjustable area of EMR incident on a surface of the tissue. The device of claim 30, wherein the modulator is electrically connected to the EMR source and is configured to vary the radiant flux emitted by the EMR source to a third value corresponding to a third tissue depth. The modulator can configured to vary the radiant flux within a continuous range, and can be configured to vary the radiant flux using a set of discrete values.

Another aspect of the invention is a device for transmitting light into tissue to treat damaged tissue or reduce pain that can include a housing having an EMR source and an aperture for allowing EMR generated by the source to pass through the housing to the tissue. The source can be configured to generate a flux of EMR passing through the aperture that is greater than or equal to approximately 0.1 W/cm².

Preferred embodiments of this aspect of the invention can include some of the following additional features. The aperture can have a diameter of at least approximately 7 cm. The device can be configured to produce a beam of EMR having a cross-sectional area of at least approximately 49 cm². The device can be configured to produce a beam of EMR having a diameter of at least 7 cm. The aperture can be adjustable, for example, from a first area configured to produce a first level of flux to a second area configured to produce a second level of flux.

Another aspect of the invention is a device for transmitting light into tissue that can have a housing having a window, an EMR source mounted within the housing, a set of optical elements mounted within the housing and forming an optical path extending between the EMR source and the window. The optical elements can be adjustable to alter a spot size of EMR emitted from the window to the surface of the tissue to alter the input flux at the surface of the tissue. The flux of the EMR emitted through the window can be greater than or equal to 0.1 W/cm2.

Another aspect of the invention is a device for treating tissue at a predetermined depth below the surface of the tissue that can have a housing having a window, and an EMR source mounted within the housing. The optical window can allow EMR to pass through the housing to the tissue surface. The EMR source can provide a level of flux corresponding to the predetermined depth and provide a power density of greater than or equal to 0.1 watts/cm2.

Another aspect of the invention is a method for irradiating tissue at depth that can include the steps of selecting a first input flux corresponding to a first tissue depth, and irradiating the tissue at the first depth using the first input flux.

Preferred embodiments of this aspect of the invention can include some of the following additional features. The step of irradiating can further include irradiating at a level that is above a minimum threshold of irradiance required to provide at least a minimally effective dose of EMR. The step of irradiating can further include irradiating at a level that is below a maximum threshold of irradiance required to provide at least a minimally effective dose of EMR. The step of irradiating can further include irradiating at a level that is above a minimum threshold of irradiance required to provide at least an effective dose of EMR and below a maximum threshold of irradiance required to provide at least an effective dose of EMR.

Another aspect of the invention is a method for treating a volume of tissue that can include the steps of irradiating a surface of the tissue with EMR having a first power density, and irradiating the surface with EMR having a second power density. The first and second power densities can correspond to a location of the volume of tissue to be treated.

Preferred embodiments of this aspect of the invention can include some of the following additional features. The power density can be modulated between the first and second power densities along a continuous curve or time-varying function (e.g., sinusoidal function), and the power density can be modulated between the first and second power densities by irradiating tissue at a set of discrete interim power densities. The method can also include modulating between the first and second power densities such that an applied power density remains above a minimum threshold of power densities that provide an effective dose of EMR to tissue at depth. The method can also include modulating between the first and second power densities such that an applied power density remains below a minimum threshold of power densities that provide an effective dose of EMR to tissue at depth.

Another aspect of the invention is a method of treating pain, which accounts for changes in a patient's condition between treatments. Specifically, patients suffering from chronic pain can have reduced sensitivity of nociceptic receptors, thus allowing for higher power settings in the beginning of a treatment course in order to maximize efficacy. As patient's condition improves during the treatment course, sensitivity of the receptors can increase, necessitating reduction in the power settings.

Another aspect of the invention involves causing a very limited irritation of the blood cells and vessel walls in the vessels of the dermis. This results in a low-grade inflammatory/growth response. Inflammatory mediators are released through the vessel walls that stimulate fibroblast activity and eventually lead to a “healing” effect.

Yet another aspect of the invention involves light-induced modification of cell responses to extrinsic stimuli. In particular, changes in the mitochondrial activity, caused by absorption of light by cytochromes, will have direct impact on variety and quantity of cytokines secreted by the affected cell.

### BRIEF DESCRIPTION OF THE DRAWINGS

Non-limiting embodiments of the present invention will be described by way of example with reference to the accompanying drawings in which:

FIG. 1 is a front perspective view of an EMR treatment system;

FIG. 2 is a front perspective view of a treatment head of the EMR treatment system of FIG. 1;

FIG. 3 is cross-sectional schematic view of the treatment head of FIG. 2;

FIG. 4 is a side schematic view of the treatment head of FIG. 2;

FIG. 5 is a schematic view of an alternate embodiment of an EMR treatment system;

FIG. 6 is a schematic view of a treatment head of the EMR treatment system of FIG. 5;

FIG. 7 is a graph showing an example of the change in the ratio of irradiance of tissue at a given depth to the flux incident on the surface of the tissue;

FIG. 8 is a graph showing an example of normalized fluence as a function of depth;

FIG. 9 is a cross-sectional schematic drawing of tissue segments that are cooled during treatment;

FIG. 10 is a graph showing skin temperature as a function of time after the on-set of exposure to EMR;

FIG. 11 is a graph showing an example of Action Efficiency of EMR in a tissue being treated as a function of fluence rate, i.e., irradiance;

FIG. 12 is a graph showing an example of the alteration of an effective treatment layer by varying (modulating) the irradiance incident on the surface of the tissue;

FIG. 13 is a graph showing an example of a waveform in which the incident irradiance is varied (modulated) in combination with a pulsed light source;

FIG. 14 is an graph showing exemplary waveforms that can be used to vary (modulate) the incident irradiance;

FIG. 15 is graphical view of an embodiment of a patient feedback mechanism;

FIG. 16 is a radiation source assembly for an EMR treatment system having two sets of radiation sources each capable of emitting radiation at a different wavelength;

FIG. 17 is a graph illustrating the bi-phasic effect of light on cell processes; and

FIG. 18 is a graph illustrating the results of three models of the depth of penetration of radiation as a function of the diameter of the beam of radiation at different parameters.

### DETAILED DESCRIPTION OF THE PREFERRED EMBODIMENTS

The devices and methods disclosed in conjunction with the embodiments discussed below provide various mechanisms to effectively irradiate and treat volumes of tissue, such as joints that lie well below the surface of the tissue. The devices and methods described below are able to effectively treat tissue located at a depth below the surface of the tissue by, among other things: delivering EMR to an active treatment area that lies deeper in the tissue than prior art methods are capable of treating; delivering an effective dose of EMR to a specific volume of tissue located below the surface of the tissue; shifting the depth at which the tissue is effectively treated over a volume of tissue needing such treatment; providing a method of pain reduction and relief, as well as a method to promote the healing of damaged tissue, by irradiating tissue with EMR in combination with controlling temperature in the target tissue region; providing a treatment regimen that adjusts the irradiance and temperature of the surface tissue to provide effective treatment of the desired, and generally sub-surface, target tissue; providing a treatment for a volume of tissue at a range of depths while maintaining the desired treatment parameters; and adjusting treatment parameters during operation based on information provided by one or more feedback or control mechanisms to maintain the desired treatment parameters throughout the volume of target tissue to be treated.

Using some or all of these features, the embodiments described are able to effectively treat a predetermined volume of tissue that lies below the surface of the skin using EMR, such as visible light or near infrared radiation, to, e.g., reduce or relieve pain and promote the healing of damaged tissue. Certain embodiments will have all of these features, and certain other embodiments can only have one or several of these features incorporated.

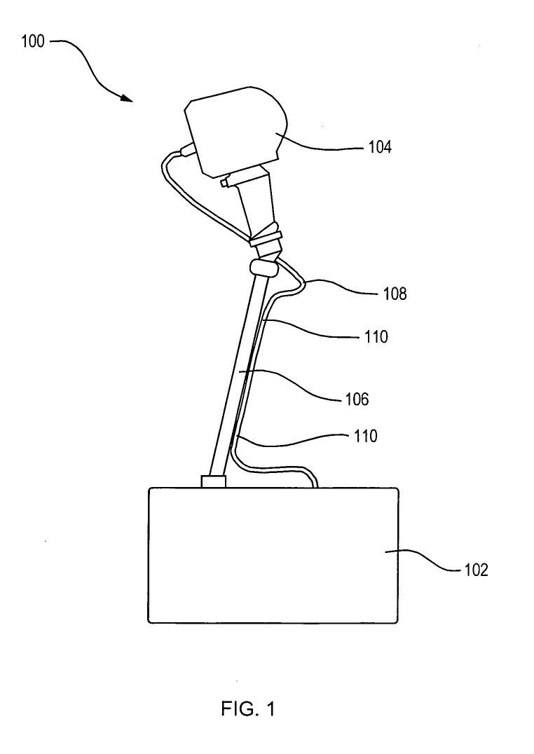

Referring to FIG. 1, an EMR treatment system 100 includes a base unit 102 and a treatment head 104. Treatment head 104 is attached to base unit 102 by a movable arm 106. Movable arm 106 also includes a set of clips 110 that secure a connector tubing 108, which extends from the base unit 102 to the treatment head 104.

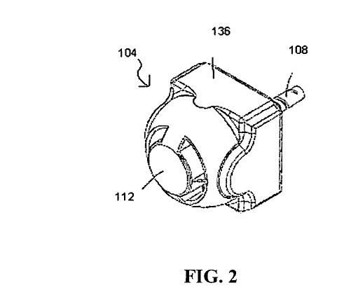

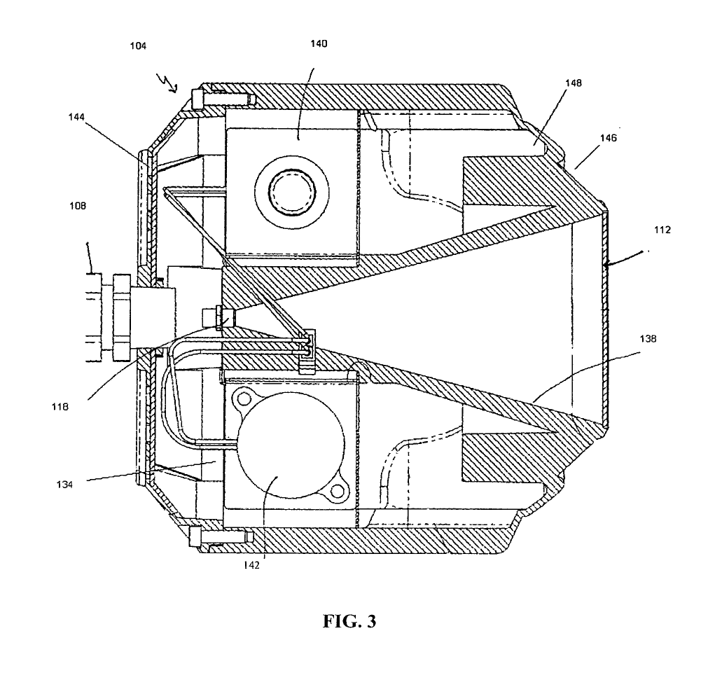

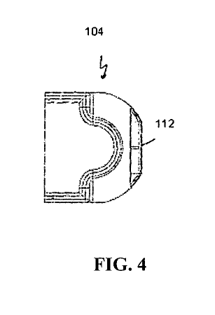

Referring to FIGS. 2 through 4, treatment head 104 includes a light source 118, an optical window 112, and a reflector 138, which are mounted in housing 136. Alternatively, treatment head 104 could include a waveguide extending between and optically coupled to a light source and an optical window, and made of an optically conductive material such as a plastic or sapphire. Reflector 138 is preferably coated with a highly reflective metal, such as a diffuse reflective white coating or a metal coating (e.g., gold, silver or copper), to maximize light delivered to the treated volume of tissue.

Treatment head 104 also includes heat exchanger 134, cooling fans 140 and 142, and cooling vents 144 and 146. Vent 144 is an input vent located on the back of treatment head 104 that allows ambient air to be drawn into treatment head 104. Vent 146 is an output vent located on the face of treatment head 104 that allows air to be ejected from treatment head 104.

EMR treatment system 100 is designed to treat a volume of tissue without placing the treatment head in contact with the skin. In other words, optical window 112 is not placed in contact with the skin during operation. Preferably the treatment head 104 will be approximately six inches from the surface of the tissue, but the device can be positioned further or closer without sacrificing performance. Optical window 112 is a plastic Fresnel lens that includes a series of ridges across the outer surface of the lens to create a constructive interference patter. The constructive interference pattern causes the lens to create a beam of EMR that is parallel and that diverges very little over a distance of approximately two feet. Therefore, the exact distance that the treatment head 104 is positioned from the tissue is not critical as long as the device is held within that maximum distance. Alternatively, the optical window can be made of sapphire, or another suitable transparent or a semi-transparent material, such as a glass.

During operation, light is generated from light source 118, which can be the terminal end of a fiber optic cable connected to an array of LED's or other light sources located within the base unit 102. The light travels from the LEDs in the base unit 102 to the treatment head 104 through the fiber optic cable extending through connector tubing 108. Alternatively, an array of LEDs or laser diodes or other light source can be located in treatment head 104. The light is then transmitted from light source 118 to the tissue to be treated via optical window 112, which is at a distance of approximately 6 inches from optical window 112. The light can travel directly from the light source 118 to the tissue or it can be reflected by reflector 138.

The cooling fans 140 and 142 pump air through the treatment head 104 both to cool the components of the treatment head 104 and to cool the tissue being treated. Air is drawn into the treatment head 104 through the vent 144, where is pumped across the heat exchanger 134 by fans 140 and 142. The air is then pumped through channels 148 and is ejected in a stream through vent 146. The stream of air is directed at the surface of the tissue being treated, and flushes excessive heat from the tissue and from the space between the surface of the tissue and the treatment head 104.

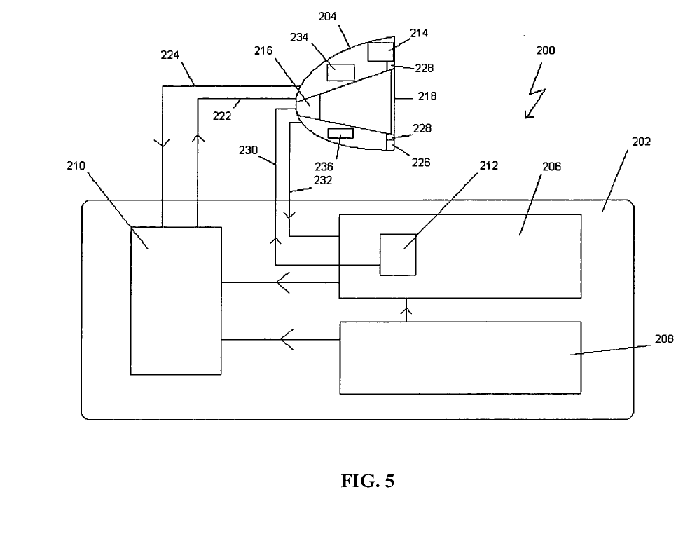

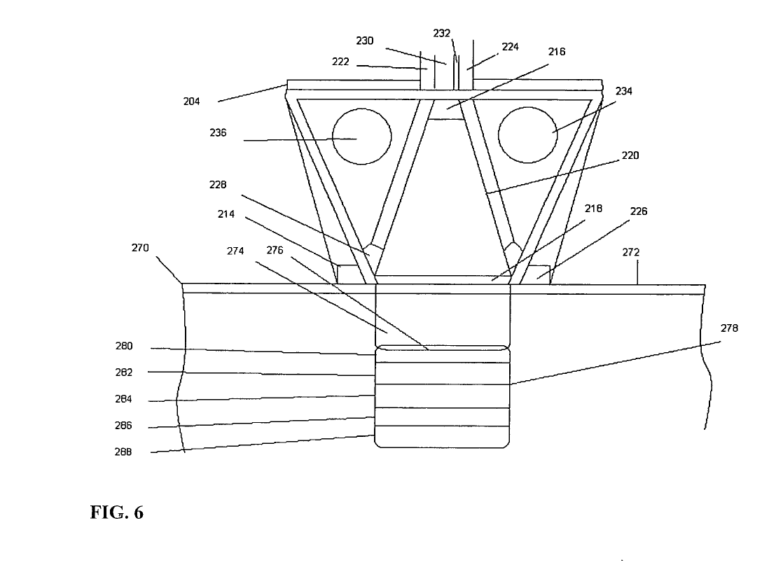

An alternate embodiment of an EMR treatment system is shown in FIGS. 5 and 6. EMR treatment system 200 includes a base unit 202 and a treatment head 204, which is designed to be in contact with and cool the surface of tissue 270 during operation. Treatment head 204 is attached to base unit 202 via a movable arm (not shown in FIG. 5) that is similar to the movable arm 106 of EMR treatment system 100. Alternatively, treatment head 204 can be connected to base unit 202 using a flexible cable that encases the connections between each. In either case, treatment head 204 could further include a handle to facilitate manipulation of treatment head 204 during operation.

Base unit 202 includes a controller 206, a power source 208, and a chiller 210. Controller 206 further includes a modulator 212, and is connected to a feedback mechanism 214, which provides a feedback signal to controller 206 via an electrical connection 232.

Treatment head 204 includes a light source 216, an optical window 218, and a reflector 220. Light source 216 is a set of LEDs, such as LEDs disposed on one or more diode bars. Alternatively, the light source could be one or more lasers, laser diodes, lamps, or any other suitable light source. Optical window 218 is a sapphire optical element suitable for the transmission of light. Light source 216 is controlled by modulator 212 via an electrical cable 230. During operation, light source 216 emits light, preferably at a wavelength of 810 nm, which travels through optical window 218 and is incident on the surface of an area of treated tissue 270.

Although both EMR treatment systems 100 and 200 are designed to emit light at approximately 810 nm, many other embodiments are possible. For example, other embodiments can emit other wavelengths of visible light as well as electromagnetic energy having wavelengths in the non-visible spectrum. Furthermore, energy outside the electromagnetic spectrum, such as radio frequencies, microwaves, and acoustic energy, including ultrasound, can be used in conjunction with particular embodiments. Additionally, energy having varying or multiple wavelengths can be employed, such as visible light having multiple wavelengths, visible light with near infrared radiation, EMR over a range of wavelengths and potentially including multiple peak wavelengths, ultrasound in conjunction with EMR, or other combinations suitable for a particular treatment. Additionally, EMR over a range of wavelengths could be used to coincide with various action spectra, for example, those disclosed in Karu et al. (which is discussed above and incorporated herein by reference) or other action spectra. Additionally, the EMR could be delivered by an array of smaller beamlets concentrated together to provide a larger beam.

Furthermore, two EMR sources could be used for different purposes, such as, for example, a first source to provide heating and a second source to provide biomodulation of the tissue. The first source could be any source appropriate to heat tissue, such as an RF source, visible light source, microwave source, or acoustic source. The first source could be used to induce hyperthermia in the tissue. The second source could be selected to provide one or more wavelengths suitable for photobiostimulation. Recently, another method of combining LILT with hyperthermia in the treated region of tissue was disclosed in U.S. patent application Ser. No. 10/680,705 entitled Methods and Apparatus for Performing Photobiostimulation (Publication No. US 2004/0162596 A1) (G. B. Altshuler, I. Yaroslavsky, M. Pankratov, D. Gal) (the “'705 application”), which is incorporated herein by reference. There, methods and devices are disclosed that utilize directed energy to control the depth of elevated temperature in the tissue.

Treatment head 204 is configured to produce a fixed spot-size, i.e., the area on the surface of the tissue on which light from an optical window 218 is incident does not vary. However, in alternate embodiments, a variable spot size could be used, for example, by including a set of adjustable optical elements between the light source 216 and the optical window 218 that control the beam size. Such a variable spot size can be used in both contact and non-contact embodiments.

Treatment head 204 is designed to be placed in contact with the skin during operation, and is capable of cooling the tissue being treated. To accomplish this, treatment head 204 further includes a cooling system. The cooling system includes the chiller 210, the optical window 218, and further includes a coolant supply tube 222, a coolant return tube 224, a temperature sensor 226, and a heat exchanger 228, which, in this embodiment, is a pathway filled with a coolant extending around the periphery of the optical window 218 to cool the edges of the optical window 218 and provide thermal diffusion of the entire optical window 218. Treatment head 204 further includes fans 234 and 236 to cool the light source and other internal components of treatment head 204.

Optical window 218 is sapphire, because sapphire provides good thermal conductivity, such that when in contact with the skin, optical window can be used to cool the tissue to be treated. Alternatively, many other embodiments are possible, including a plastic window similar to optical window 112 of EMR treatment system 100 or an open window with to plate or covering extending across the opening. Similarly, the cooling mechanism could be any suitable cooling mechanism able to reduce the temperature of the optical window and/or the tissue being treated.

Temperature sensor 226 monitors the temperature of the optical window 218 to monitor cooling and provide control signals to the controller 206. Alternatively, a temperature sensor could, instead of or in addition to temperature sensor 226, be configured to directly measure the temperature of the tissue being treated.

Alternatively, cooling systems can use air or other suitable gas that is blown over the cooling surface, or cooling oil or other fluid. Also, a water or refrigerant fluid (for example R134A) spray can be applied to the optical window 218 or the coolant can be applied across the entire surface of the optical window 218. Mixtures of substances, such as an oil and water mixture, can also be used. In an alternate embodiment, the cooling system can be a series of tubes that carry a coolant fluid or a refrigerant fluid (for example, a cryogen), which tubes are in contact with tissue 270 or are contained within an optical window. In yet another embodiment, the cooling system can include a water or refrigerant fluid (for example R134A) spray, a cool air spray or air flow across the surface of tissue 270. In other embodiments, cooling can be accomplished through chemical reactions (for example, endothermic reactions), or through electronic cooling, such as thermoelectric cooling.

In yet other embodiments, the cooling system can have more than one type of coolant, or the cooling system can not include a contact window or plate, for example, in embodiments where the tissue is cooled with a cryogenic or other suitable spray directly applied to the tissue. In other embodiments, two or more cooling mechanisms can be included in the same device. For example, one cooling mechanism can be used to cool the light source and a second cooling mechanism can be used to cool the optical window and tissue.

Furthermore, many alternate embodiments of the treatment head are possible. For example, the base unit could be eliminated and all of the control circuitry could be included in the treatment head to create a stand-alone device, such as a handpiece or other device. Similarly, a device could be configured to be operated by a consumer in the home or other non-medical environment.

In other embodiments, an array of LEDs could be provided to create a beam of EMR. The LEDs could be part of a light source assembly using an optical window similar to the configuration of treatment heads 104 and 204, or they could be provided essentially at the same relative location as the optical window in treatment heads 104 and 204, thereby approximating the performance of a fully filled aperture using a single light source. In such embodiments, the LEDs could provide various light intensities by powering only a portion of the LEDs at a time. When the LEDs are configured to essentially fill the area of the optical window or aperture, the device can not be able to achieve a 100% “fill factor” as compared to a beam that is formed using a light source assembly that emits EMR through an optical window spaced some distance away, as in treatment heads 104 and 204. The maximum fill factor, measured as a percentage of a fully filled beam, that can be achieved by such a device is dependent on the spacing and density of the LEDs.

EMR treatment systems 100 and 200, as well as many alternate embodiments, can be used to reduce chronic and acute pain, as well as promote the healing of damaged or wounded tissue, using non-invasive methods. To effectively treat a predetermined volume of tissue at depth, the embodiments described herein incorporate one, some or all of the following features:
1. EMR can be transmitted at a higher level of irradiance at the surface, i.e., a higher level of input flux, than prior art devices and methods used for treating pain or healing tissue;
2. EMR can be transmitted within a relatively narrow band or range of irradiance levels, and can be preferably delivered at the maximum Action Efficiency;
3. The input flux of EMR incident on the surface of the tissue is modulated to control the depth at which tissue is effectively treated;
4. Cooling can be applied to the tissue near the surface;
5. The spot size of the EMR that can be incident on the surface of the tissue can be large enough to prevent lateral beam degradation due to scattering; and
6. A feedback mechanism controls the irradiance level to account for changes in tissue composition, such as results from increased blood flow.

### Transmission of EMR at Higher Levels of Input Flux

The depth of penetration of EMR into the tissue being treated is dependent in part on the input flux. The higher the input flux, the deeper the EMR will penetrate into the tissue. The level of irradiance of EMR at a given depth is attenuated as it penetrates the tissue, and increasing the input flux causes the EMR to penetrate deeper into the tissue being treated.

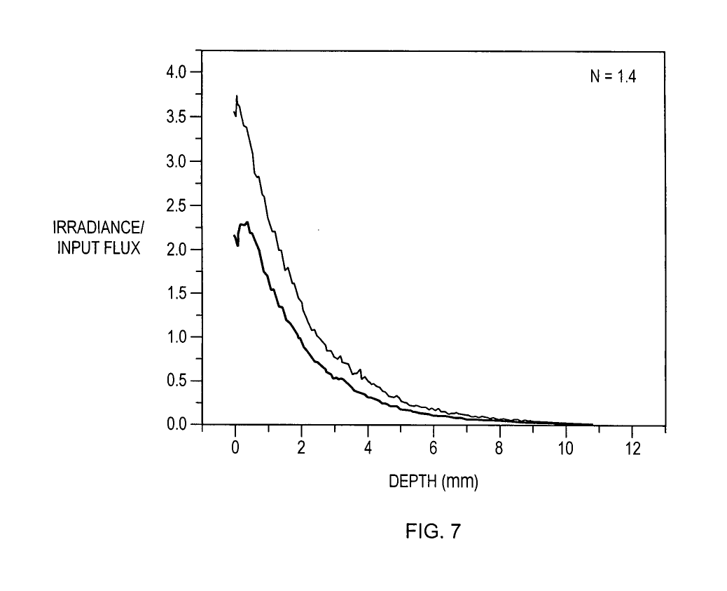

FIG. 7 provides an example of how the level of irradiance decreases as EMR penetrates tissue. The horizontal axis of the graph in FIG. 7 provides the depth in the tissue in millimeters. The vertical axis provides the ratio of irradiance at depth to the input flux at the surface. Irradiance is a measure of the power density of EMR that is transmitted to an area of tissue below the surface of the tissue, and is measured in, e.g., W/cm². The irradiance at depth is not directional, i.e., the EMR can be incident on a given volume of tissue from any direction, and can be the result, e.g., of scattering and other phenomenon. Input flux is a measure of the power density of EMR that is incident on the surface of the tissue and is measured in, e.g., W/cm². Input flux is directional and is the measure of EMR incident on the surface of the tissue and emitted from the treatment device.

As shown in FIG. 7, the ratio of irradiance to input flux decreases with depth. millimeters. The upper curve corresponds to Type II skin, which is average Caucasian skin and the lower curve corresponds to Type VI skin, which is average African American skin (i.e., more melanin is present in African American skin than Caucasian skin). In other words, in the cases presented, where the input flux (the denominator in the ratio) remains constant and the overall ratio decreases with depth, the level of irradiance (the numerator in the ratio) decreases with depth.

The ratio of the irradiance at depth to the input flux is greater than one at the skin surface (depth of 0 mm) until a depth of approximately 2 mm due to back scattering of EMR from the surrounding tissue. This results in a concentration of EMR at the surface. When the light penetrates several millimeters into the skin, the ratio drops off quickly, indicating that the irradiance decreases quickly at depth.

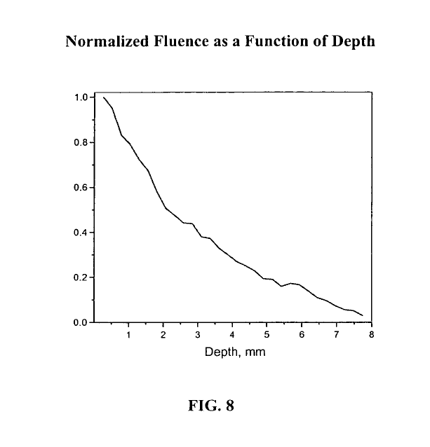

FIG. 8 illustrates a related concept, i.e., that fluence of EMR (in this case, visible light having a wave length of 810 nm) decreases with depth into the tissue being irradiated. The graph in FIG. 8 has a horizontal axis representing depth in tissue in millimeters and a vertical axis representing normalized fluence. Fluence is the amount of energy per unit area measured in, e.g., J/cm². Normalized fluence is a representation of the fluence where the value has been normalized to a value of one at the surface, with subsequent measurements shown relative to that starting value. The graph of FIG. 8 was obtained from Monte Carlo simulations. (Ripples in the curve are caused by statistical nature of the technique).

FIGS. 7 and 8 illustrate that, as the depth increases, the amount of light that penetrates the tissue is attenuated. The attenuation is a result of absorption and scattering in the skin and subcutis. These graphs also demonstrate that, when a relatively higher level of input flux is applied at the surface of the tissue, a relatively higher level of irradiance penetrates to the various depths within the tissue.

To provide treatment at greater depths in treated tissue, EMR treatment systems 100 and 200 transmit EMR at a relatively high level of input flux. For example, the light transmitted by EMR treatment system 100 is preferably 800 to 850 nm delivered at an input flux of approximately 0.1 to 1.5 W/cm². Although, as discussed below, the range of values for input flux will vary depending on the parameters of each particular treatment.

The input flux is on the order of approximately 10 times greater than what has typically been used in existing treatments for pain relief and wound healing. For example, EMR treatment system 100 is capable of delivering approximately 70 J/cm² of energy using optical window 112, which has an area of approximately 50 cm². Treatment head 104 is capable of providing a radiant exposure that is >30 J/cm2 and a power of approximately 20 W. Typically, EMR treatment system 100 irradiates the surface of the tissue being treated with EMR having a power density in the range of approximately 4 W/cm² to 10 W/cm². In comparison, to date, most of the pain-treating light devices have been under a specific power level of 1-3 W (considered to be threshold for thermal effects) and usually between 5 mW and 100 mW. (By comparison, a laser pointer provides approximately 2-3 mW.) EMR treatment system 100, therefore, can achieve relatively deeper penetration of light and other EMR.

EMR treatment systems 100 and 200 can be used, for example, to treat a joint that lies at a depth that is at a greater distance from the surface than what light at lower power densities will penetrate. Thus, EMR treatment systems 100 and 200 can treat pain and/or damaged tissue in, for example, a shoulder, knee or hip joint.

### Transmission of EMR within a Range of Irradiances to Achieve Maximum Action Efficiency

Most research and existing treatments for pain have presumed that the EMR fluence (i.e., the energy applied to an area of tissue, e.g., J/cm²) of the EMR was the critical parameter. Relatively little consideration has been given to the effect of the rate at which the EMR fluence is transmitted to the tissue. In other words, most existing research focused on the total dose of EMR that was applied to a given area of the tissue, and not on the overall rate at which the dose was applied. As a result, many treatments and studies have utilized low power levels over longer periods of time to achieve the desired dose of light.

However, such treatments result in a limited photon density (proportional to irradiance) at deep tissue areas, limiting the effective penetration depth of the EMR. As a result, the effectiveness of such treatments is often limited to treating tissue near the surface of the tissue. A treatment will not be effective, if it attempts to treat tissue using an input flux that is too low. The input flux of the EMR is an important treatment parameter. It affects both the depth of penetration and, as discussed below, the effectiveness of the treatment. For example, several studies and other publications have determined that the Bunsen-Roscoe law of reciprocity does not hold for many light-induced tissue effects. The law of reciprocity states that a certain biological effect is directly proportional to the total energy dose irrespective of the rate at which the dose is applied.

As the irradiance of EMR at depth within tissue being treated decreases, the treatment can become ineffective. As discussed above, the level of irradiance within the tissue at a given depth is related to the input flux. Thus, to apply an effective dose of EMR to a volume of tissue at a given depth, the proper input flux must be used to ensure that the level of irradiance within the target volume is appropriate to deliver an effective dose of EMR.

An effective dose of EMR is delivered to tissue at a given depth when the level of irradiance falls within a specific range. If the level of irradiance is too high or too low, the effectiveness of the treatment is greatly reduced and the treatment can not be effective at all.

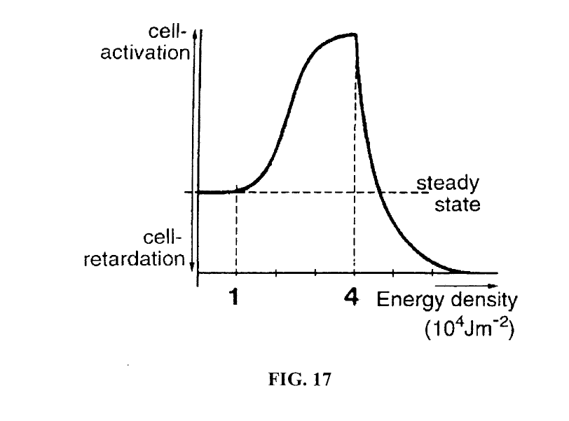

Referring to FIG. 17, the bi-phasic effect of light and other EMR on cells and the healing process has been the subject of recent study. (See, e.g., Sommer, Andrei P., et al., “Biostimulatory Windows in Low-Intensity Laser Activation: Lasers, Scanners, and NASA's Light-Emitting Diode Array System”, Journal of Clinical Laser Medicine & Surgery, Vol. 19, No. 1, p. 29-33, 2001.) The graph in FIG. 17 is an Arndt-Schultz curve demonstrating that the effect of EMR on cell processes (e.g., mitosis) generally appears to be a function of the energy density applied. Specifically, a given cell process appears to be activated and/or modulated within a range of intensities of the EMR that is applied. The resulting process tends to increase as the intensity of light or other EMR increases. Below a minimum threshold of EMR intensity, there typically will be no response, little response, or an insufficient response. Above that minimum threshold, the effect or process will increase until it reaches an apex somewhere above that minimum threshold. After reaching that relative maximum, the resulting cell process tends to decrease as the intensity of light or other EMR continues to increase, and, as shown, can decrease rapidly. Above a maximum threshold intensity of EMR, there typically will be no response, little response, or an insufficient response. Thus, to promote a given process, it can be preferable to treat within these limits.

Further, as illustrated in FIG. 17, as the energy density continues to increase, the cell processes can actually be inhibited. Thus, higher intensities can be used to suppress or switch off various cell processes.

These principles can be applied beyond the modulation of cell processes and used to facilitate and more effectively treat pain, promote healing, and/or reduce scarring.

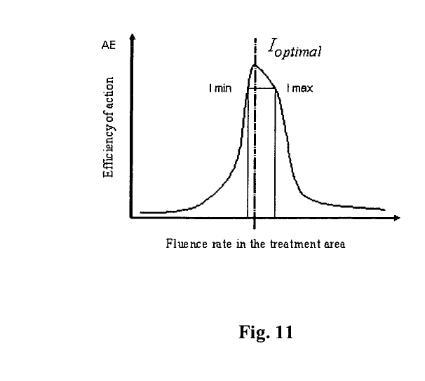

Referring to FIG. 11, to effectively treat tissue at a given depth, the level of irradiance of the EMR is kept within a specific range of irradiances. In FIG. 11, the vertical axis represents efficiency of action or Action Efficiency (“AE”), which is a relatively measure of the effect that the energy applied to the tissue has on the tissue. The horizontal axis represents the irradiance, i.e., the fluence rate, within the tissue. When tissue is irradiated within that relatively narrow band of irradiances (between Iₘₐₓ and Iₘᵢₙ), the effectiveness of the treatment on that tissue, i.e., the Action Efficiency, is at its highest, with the maximum AE (AEₘₐₓ) occurring within the range at the optimal level of irradiance (Iₒₚₜᵢₘₐₗ). When the tissue is irradiated at levels above or below that range, i.e., above or below the thresholds Iₘₐₓ and Iₘᵢₙ, the AE of the treatment quickly decreases. When the level of irradiance is too far above or below the range, the EMR has essentially little or no effect on the tissue and the AE is too low to be considered significant.

In particular, efficiency of the light treatment has a sharp maximum at the level of irradiance corresponding to Iₒₚₜᵢₘₐₗ. Depending on the wavelength, the particular mechanism of action, and tissues involved, this maximum can be in the range between 0.1 and 100 mW/cm², preferably between 0.5 and 50 mW/cm². As seen from the attenuation plot of light in tissue shown in FIG. 8, the dependence of the Action Efficiency on the level of irradiance restricts the effective treatment volume to a relatively small layer of tissue, if the input flux is kept constant.

As FIG. 11 illustrates, it is desirable to treat tissue within the irradiance band at which the AE is highest. Outside that range, the AE quickly drops, and the dose of EMR delivered is much less effective and, depending on how far outside that range, can not be effective at all. The boundaries of the irradiance band will vary depending on various factors, including the wavelength of the EMR used, the type of tissue being treated and the depth of the tissue. (FIG. 11 is exemplary only, and is not intended to define the irradiance band that would be preferable for all types of treatments.)

### Modulation of the Flux Incident on the Surface of the Treated Tissue

As discussed above, for a given input flux at the surface of the tissue being treated, both the fluence and the irradiance delivered within the tissue vary with depth. Thus, to effectively treat an entire volume of tissue at depth, the input flux can be adjusted to ensure than an effective dose of EMR is delivered to the tissue throughout the entire volume being treated, i.e., at each depth within the tissue volume.

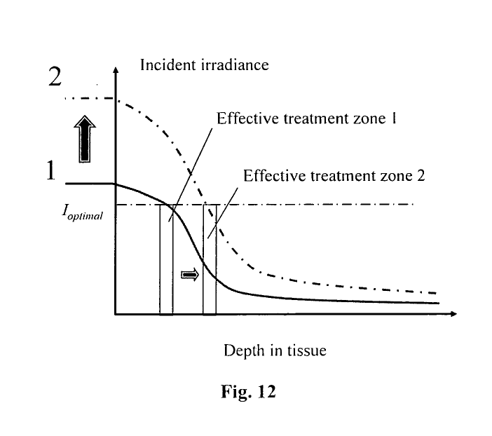

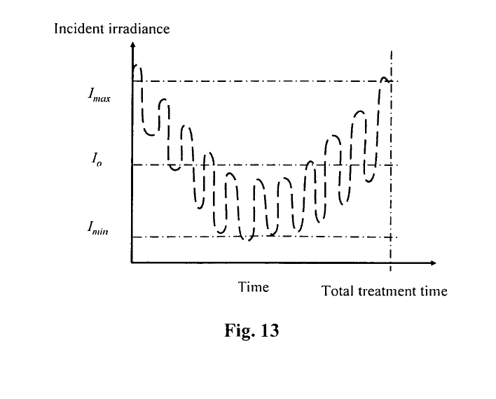

Referring to FIGS. 12 and 13, the input flux can be modulated to control the depth at which the tissue is effectively treated. By altering the input flux, the depth to which the EMR penetrates into the tissue is altered. As shown in FIG. 12 and as discussed above, increasing the input flux (labeled incident irradiance on the vertical axis of FIG. 12) causes the EMR to penetrate more deeply into the tissue. Thus, when the input flux is increased from input flux level 1 to input flux level 2 in FIG. 12, the level of irradiance is higher at each depth in the tissue. In other words, increasing the input flux changes the curve that defines the level of irradiance as a function of depth.

Therefore, the depth of the tissue that is being treated by an effective dose of EMR at a given time can be varied and controlled by modulating the level of irradiation. Assuming that the composition of the tissue in the volume is uniform, the optimal irradiance will not change. Therefore, by increasing the input flux, the effective treatment layer between Iₘₐₓ and Iₘᵢₙ is shifted deeper into the tissue, and a different volume of tissue is treated. (Note, if Iₒₚₜᵢₘₐₗ does vary, because, for example, the tissue composition is not uniform or due to some other factor, the change can be compensated by adjusting the treatment parameters accordingly.)

Generally, when the magnitude of the input flux is larger, the depth of the tissue (Z) that is effectively treated is greater, i.e., the effectively treated tissue is relatively deep. On the other hand, generally, when the magnitude of the flux is smaller, the depth of the tissue that is effectively treated is less, i.e., the effectively treated tissue is relatively shallow. The input flux, therefore, determines the depth of treatment. By modulating the flux incident of the surface, the function of irradiance delivered as a function of depth changes. In other words, as shown in FIG. 12, by increasing the flux, the delivered irradiance as a function of depth changes from curve 1 (solid line) to curve 2 (dashed line). Therefore, the depth at which the optimal irradiance is delivered changes.

The magnitude of the flux can be altered to correspond with the boundaries of a volume of tissue to be treated. By varying the flux over a range of magnitudes, an entire predetermined volume of tissue can be treated corresponding to the surface area of the tissue that is irradiated and lying between the maximum and minimum depths of tissue that is treated with an effective level of irradiation. This can be done, preferably, by gradually increasing the input flux from a first value corresponding to the shallowest layer of the treatment volume to a second value corresponding to the deepest layer. Other alternatives are possible, including decreasing the value of the input flux or using a set of discrete values of input flux between the maximum and minimum input fluxes.

Using these principles, specific tissue volumes at depth can be targeted and treated. For example, a treatment can treat a shoulder joint by first irradiating the tissue at a level that effectively treats the tissue, and varying the flux of that irradiation at a magnitude that corresponds to the depths at which the should joint is found. As another example, referring to FIG. 6, by varying the flux to ensure that the proper dose of EMR is delivered at predetermined depths within the tissue, an entire volume can be treated. An entire volume 278 is treated by sequentially treating a series of sub-volumes 280-288 within the tissue.

Referring to FIG. 13, the modulation can be combined with the pulsed mode of treatment. The modulated curve preferably is smooth enough to provide uniform coverage of the desired treatment volume. The entire range of effective irradiance (i.e., between the thresholds Iₘₐₓ and Iₘᵢₙ) is shifted deeper into the tissue. In this regime, pulsing frequency is typically higher than the modulation frequency. For example, EMR treatment system 200 transmits EMR as pulses having a duty cycle of 1.33 sec., in which the LED array is on for approximately 1 sec and off for approximately 0.33 sec.

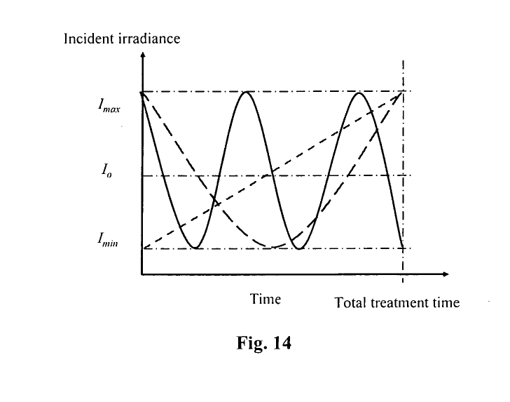

In effect, to extend the volume of effectively treated tissue, the incident irradiance is modulated in time, providing scanning of the desired treatment volume. The modulation function that is used can be an aperiodic or periodic function. Referring to FIG. 14, many functions are possible for modulating the flux at the surface of the tissue. Three such waveforms are shown in FIG. 14, but many more are possible. However, preferably, the function has a gradually increasing or decreasing curve, such as a sine wave or other waveform. Although other functions, such as a step function, can be effective, they can not be as effective in treating tissue as a function that changes gradually.

Preferably, the modulation function is a harmonic function with a frequency between 0.01 and 1 Hz. The modulation function is characterized by the mean incident irradiance I₀ and by the modulation depth

$$M = \frac{{I}_{max} - {I}_{min}}{2 {I}_{0}} ,$$

where Iₘᵢₙ and Iₘₐₓ are minimal and maximal values of the incident irradiance. The mean incident irradiance is preferably in the range between 50 and 5000 mW/cm² (although other ranges are possible), and the modulation depth can typically be in the range 0.1 to 1 mm.

To determine the precise dimensions of the volume to be treated, a diagnostic tool, such as an x-ray or CAT scan can be employed. To determine the parameters of the treatment, the controller can perform a Monte Carlo calculation or, preferably, refer to a look-up table to obtain information regarding such calculations. Alternative methods are also possible, including interfacing information from a three dimensional imaging device to provide data to the EMR treatment device, which can be analyzed to determine the treatment parameters.

Other embodiments of the invention are capable of determining the treatment parameters in real time using sensors that provide data to the controller that determines and adjusts the treatment parameters during the treatment. Such embodiments preferably include controllers having memory, and processing capability. For example, an EMR treatment system according to the invention can include a microprocessor or a personal computer or have attachments that allow the system to be connected to a personal computer, a computer network, other types of computers and/or other types of medical equipment.

### Cooling of Surface Tissue

By cooling the tissue at the surface, the effective treatment volume can be pushed deeper into the tissue. The depth of photobiostimulation can be extended by applying a combination of directed energy and surface cooling to create controlled hyperthermia in desired (generally speaking, sub-surface) regions of tissue.

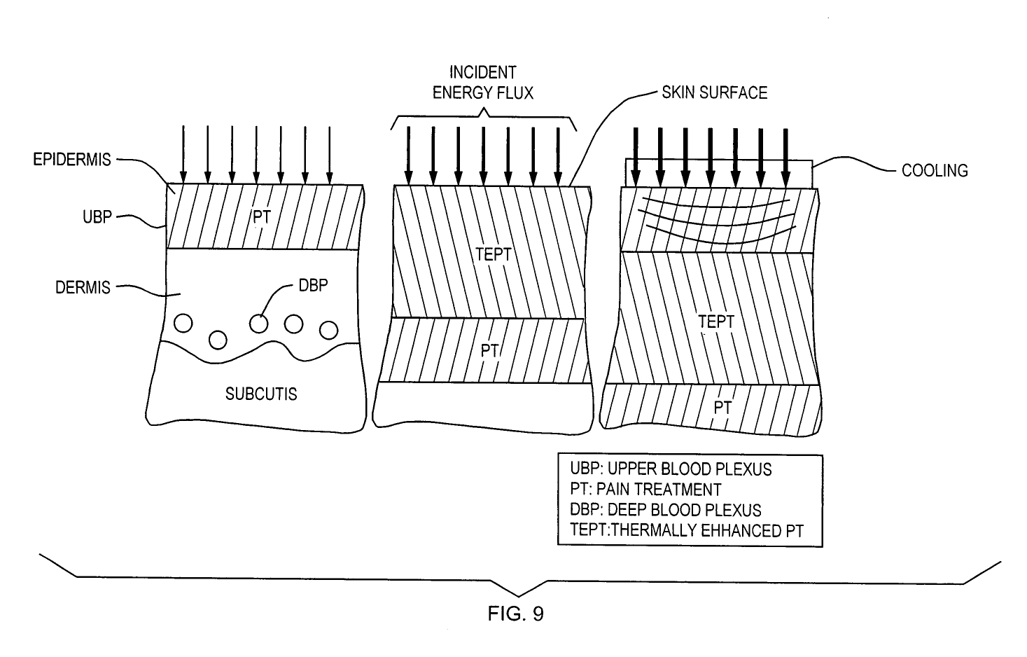

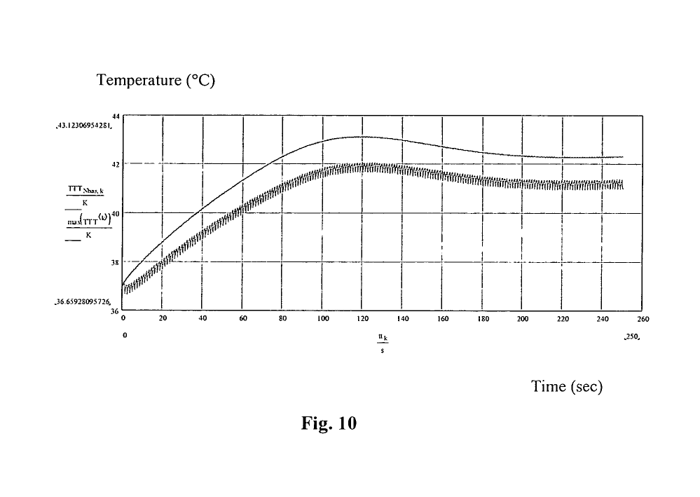

For example, referring to FIG. 9, to better control the dimensions of the volume of tissue that is treated as well as the overall depth of the volume of tissue that is effectively treated, the surface of the skin can be cooled to optimize the temperature profile within the tissue. Human tissue is typically about 37° C. The temperature of approximately 45° C. is a threshold of irreversible damage to cells. An example of the temperature profile associated with exposure to EMR is shown in FIG. 10, which illustrates the calculated dynamics of skin temperature as a function of time after on-set of the exposure to EMR. The upper curve indicates the maximum skin temperature, and the lower curve indicates the temperature at the basal layer of the epidermis (approximately 100 μm in depth).

By cooling the surface tissue, the destruction of the tissue at and near the surface can be prevented. The temperature of the skin at or near the surface is lowered to counter the heat generated within such tissue by absorption of light that passes through that surface tissue as it is transmitted to the deeper tissue to be treated.

Therefore deeper tissue can be treated without thermal damage to the tissue closer to the surface. By cooling the surface tissue and the subsurface tissue directly below the surface, the volume of effectively treated tissue can be deeper than without cooling. A relatively higher input flux can be used so that the volume of effectively treated tissue is relatively deeper. However, the layer of cooled tissue at the relatively shallower depths near the surface can withstand the higher levels of irradiance near the surface without overheating. Thus, the shallower tissues are not damaged.

By simultaneously employing contact cooling of skin surface, the resulting hyperthermia can be advantageously shifted to the desired depth in the body, thus inducing thermally-enhanced photobiostimulation at selected locations. By way of example, EMR treatment system 200 can be used to provide a desired temperature profile throughout the tissue being treated by cooling the surface tissue to a desired level. Referring to FIG. 6, during operation of EMR treatment system 200, heat energy can be drawn from tissue 270 across optical window 218, where it is transferred to coolant contained in the cooling system via the heat exchanger 228. Here, the coolant is water chilled to a temperature between approximately 5° C. and 25° C. by circulating the coolant through chiller 210.

The cooling system can be used to reduce the surface temperature of tissue 270 from its normal temperature, which can be, for example, 37° C. or 32° C., depending on the type of tissue being treated, and can be higher during treatment due to the heating of tissue by the emitted EMR. The cooling applied to surface 272 of the tissue reduces the temperature of a cooled tissue volume 274 that lies just beneath the surface 272. To obtain the desired temperature profile in cooled tissue volume 274, the cooling system cools optical window 218 to approximately 5° C. (i.e., approximately the same temperature as the chilled water), resulting in a tissue temperature of between approximately 5° C. and 32° C. at the surface 272 and between approximately 20° C. and 37° C. at the lower boundary 276 of the cooled tissue volume.

In other embodiments, a cooling system can be used to decrease the temperature of the surface of tissue 270 to other temperatures, for example, to a temperature within a range between 25° C. and −5° C. The exact temperature will depend on the treatment. More cooling will be desired when higher irradiances are used to penetrate more deeply into the tissue. Other factors, such as the type of tissue being treated, will also affect the amount of cooling required at the surface of the tissue to achieve the desired temperature profile. Thus, the treatment parameters can vary between treatments.

### Large Spot Size/Beam Size

In addition to the surface flux, the spot size or beam size of the treatment device also affects the irradiance delivered to the tissue volume at depth. A larger beam size helps minimize the effects of scattering when the EMR strikes and/or penetrates the tissue being treated. Multiple scattering events attenuate the propagation of light. When the effective scattering coefficient is known, however, the changes caused by scattering can be corrected. Due to the amount of scattering within the tissue, a narrow beam is quickly diffused when it interacts with the tissue. Thus, a narrow beam typically cannot penetrate beyond a few millimeters below the surface of the tissue. The EMR becomes highly diffuse quickly as the EMR interacts with the tissue, and the intensity of the beam decreases below the limits that are effective for treatment.

By using a larger beam size, the attenuation of the irradiance at depth that is caused by scattering is reduced. By way of example, for a small diameter beam of, e.g., approximately 1 mm, the mechanism of attenuation is primarily scattering (as opposed to, e.g., absorption). This results in a 1/e distance of approx. 0.1 mm. For a wide beam, e.g., 10 mm or greater, the mechanism of attenuation is mostly absorption, which, at 800 nm, results in a 1/e distance of approx 1 mm. Thus, the wider beam penetrates the tissue to approximately ten times the depth of the narrower beam, within limits dependant on the, e.g., the type of tissue and other factors.

Although scattering still occurs in the wider beam, the scattering occurs throughout the beam. Therefore, some of the light will be scattered from the outer periphery of the beam, thereby attenuating the irradiance at the edges. However, within the periphery of the beam, light will be scattered from one portion of the beam to another, and the attenuation due to scattering will be reduced.

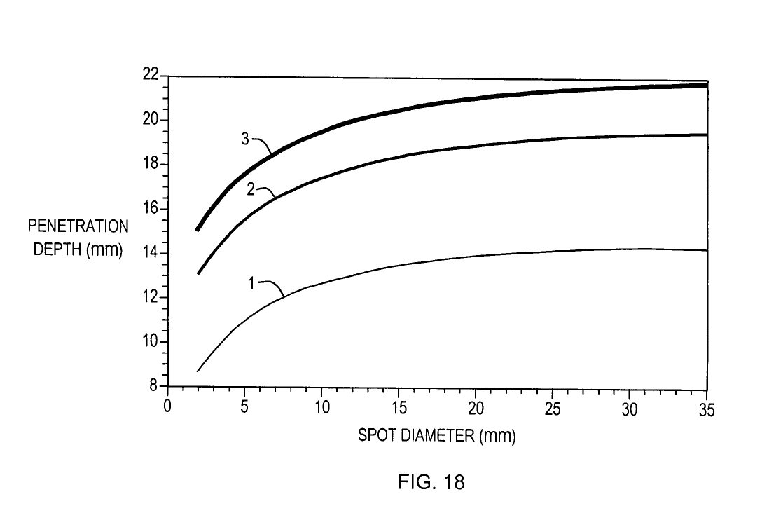

An example of the relationship of beam diameter to penetration depth is shown in FIG. 18. The three functions of FIG. 18 were created using a computer model of the optical properties of skin that, among other things, approximates the optical properties of skin tissue. In the diffusion model, three cases were simulated using a wavelength of 810 nm and skin of type II. The model also presumed that the beam profile was flat across the beam, and that the light was applied through sapphire with a normal incidence. The input flux for each curve in FIG. 18 is shown in Table I below. The graph shows the penetration depth for each case as a function of beam diameter. (Though a circular beam is presumed in the model, similar results would be obtained for beams having other cross-sectional shapes and areas.) The penetration depth for each case is the deepest depth where the bulk irradiance (in the direction of the beam) is above a threshold value for stimulating biochemical activity, which is defined for purposes of each case shown in FIG. 18 as 5·10⁻³ W/cm². However, that threshold can be different in different subjects, in different types of tissues, and for different applications. Further, different thresholds of irradiance can be pertinent in other embodiments of the invention.

TABLE I
Values Used In Simulations Illustrated In FIG. 18

| | Input Flux, | Threshold of Bulk |
|---|---|---|
| Curve | Watts/cm² | Irradiance, W/cm² |
| 1 | 0.1 | 5 · 10⁻³ |
| 2 | 0.5 | 5 · 10⁻³ |
| 3 | 1 | 5 · 10⁻³ |

FIG. 18 is a graph showing the relationship between penetration depth (along the vertical axis) and beam diameter (along the horizontal axis). The maximum penetration of radiation is the limit of the penetration at a hypothetical device having an infinite diameter. These three curves 1-3 demonstrate that the depth of penetration can be varied by varying the diameter of the beam of radiation that is applied. The curves 1-3 also illustrate that a larger beam is more effective in delivering radiation to a deeper depth than a relatively narrower beam. Thus, beam diameter can be combined with other variables such as surface flux to, among other things, achieve treatments at various depths and to vary the depth of penetration to treat a volume of tissue. Note that each curve approaches a limit of penetration depth, which is corresponds to a hypothetical infinite beam diameter. This demonstrates the limit on depth penetration that can be achieved by varying the beam diameter. Note that, although there is a hypothetical limit of depth of light penetration that can be achieved for a given set of parameters, that limit will vary as other parameters are varied, e.g., input flux.

The larger beam size has the advantage of increasing the depth to which the EMR will penetrate the tissue to deliver an effective dose of EMR. Additionally, in some circumstances, it will be capable of simultaneously treating multiple-trigger points in the tissue volume, i.e., multiple sources of pain that can be located within the area treated by the beam. Also, the larger beam size will allow a treatment to be performed more quickly, and, thus, can have an economic advantage. However, as the size of the beam increases, more energy is required to maintain the power density, which can increase the cost and size of the device.

Preferably, a beam will be greater than 7.0 cm in diameter to further increase depth of penetration of the EMR and maintain the desired level of irradiation. The larger beam size also allows faster treatments of large areas, and provides simultaneous treatments of several trigger points. However, smaller beam sizes, though potentially less effective, can be used depending on the requirements of the particular treatment. The beams produced by treatment heads 104 and 156 are circular and have an area of approximately 50 cm².

In order to minimize both scattering and absorption of the applied optical radiation, the EMR produced preferably has a wavelength which is minimally scattered and absorbed, the available wavelengths decreasing with increasing depth as generally indicated in Table II below. The longer the wavelength, the lower the scattering; however, outside of the indicated bands, water absorption is so high that little radiation can reach tissue at depth.

In other embodiments, the beam size can be adjusted to various sizes to control the depth at which tissue is effectively treated. Similarly, devices having static beam sizes, can have larger or small beam sizes depending on the application. Alternate shapes of the area in which EMR is incident on the surface of the tissue can also be used.

### Control System Feedback

A feedback mechanism can be devised that ties the flux at the surface of the tissue to the desired modulation of the treatment for different tissue types. For example, ultrasound could be used to determine the underlying structure of the tissue. Similarly, Optical Coherence Tomography, such as Optical Diffuse Technology (ODT) or Optical Doppler Imaging (ODI), could be employed as part of the feedback mechanism. Such a feedback system would look for an increase in blood flow and compensate for the change. Thus, the system would be able to compensate for increased blood flow to the treated tissue area. As blood flow increased within the tissue, the system would adjust to account for the change in tissue composition resulting from the increased blood flow within the treated tissue volume.

Referring to FIGS. 5 and 6, EMR treatment system 200 includes feedback mechanism 214, which is an ODI sensor that measures the rate of blood flow in the tissue. As treatment begins, exposure to the EMR causes hyperthermia in the tissue. The natural response of the body is to increase blood flow to the heated tissue. Feedback mechanism 214 measures the relative increase in blood flow, and transmits a signal to the controller 206 indicating the change. The controller then recalculates any changes in the treatment parameters based on the change in overall composition of the tissue, due to the greater percentage of blood flowing within the tissue. For example, the controller can account for increased cooling by the body resulting from the blood flow through the tissue. Furthermore, the controller can alter the value of optimal irradiance based on the change in composition of the tissue, and it can alter the treatment time. Many other embodiments are possible.

In other embodiments, feedback sensors can be incorporated that provide real time feedback that can be used to adjust the various treatment parameters, based on variation in the value of Iₒₚₜᵢₘₐₗ or due to changes in other relevant conditions and/or parameters. For example, sensors that measure various parameters such as tissue temperature, surface reflectivity, surface irradiance, tissue composition, etc. can be integrated with a control system to provide real time feed back and set and adjust treatment parameters during treatment. A radiometer could be employed to measure surface reflectivity in a device.

The following parameters can have a bearing on determining the source of pain in the tissue, or provide information regarding the optical path from the skin surface to the likely pain source volume (PSV): skin surface temperature, rate of change of skin temperature, skin pigmentation (pigmentation index), incident radiant flux (which can be measured using a radiometer at the skin surface), blood velocity (which can be measured using Doppler velocipede), composition of optical characteristics of the tissue between the skin surface and the PSV (which can be measured using x-ray, ultrasound, or other means). These and other parameters can be measured using appropriate sensors integrated with a control system in various embodiments.

Other embodiments would preferably include a set of look up tables of information concerning the various treatment parameters, to ensure that processing is timely during treatment, and that potentially time-consuming calculations, such as Monte Carlo calculations, are not necessary during treatment.

Additionally, other feedback mechanisms can be included in connection with EMR treatment systems 100 and 200 as well as other embodiments. For example, a patient feedback mechanism can be included. Since the desired treatment volumes can differ from one individual to another, efficacy of treatment can be increased by allowing individual adjustments of treatment parameters during treatment. In some embodiments, this can be achieved by providing the patient with a feedback mechanism. Preferably, the feedback mechanism should include control of at least one (and, more preferably, both) of the mean incident irradiance and the modulation depth.

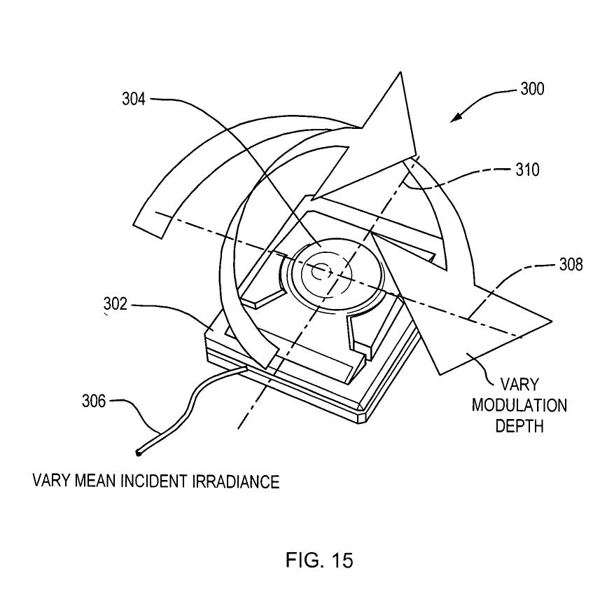

Referring to FIG. 15, an exemplary embodiment of a human interface feedback device 300 is shown. Feedback device is a trackball-type device that includes a main housing 302 and an input mechanism 304, which in this case is a rotating ball that is secured in the main housing 302. Information from the feedback device 300 is transmitted to an EMR treatment device via an electrical connection 306.

Feedback device 300 allows a patient to subjectively assess the efficacy of pain reduction during treatment and adjust the parameters accordingly by manipulating the input mechanism 304. If the input mechanism 304 is rotated in a lateral direction 308, the modulation depth is adjusted. If the input mechanism 304 is rotated in the longitudinal direction 310, the mean incident irradiance is varied. The associated EMR treatment device can store the individual optimal parameters and retrieve them during subsequent treatment sessions. The control system of the EMR treatment device, however, preferably governs any changes input by the patient, e.g., to prevent potential harm during treatment and ensure that the treatment is effective. Other embodiments of the feedback mechanism are possible, and would preferably allow for variation of the modulation frequency.

In other embodiments of the invention, the feedback mechanism can rely on instrumental means rather than on subjective input by the patient. This can be achieved, for example, through monitoring the nociceptic activity in the treatment area through either electrical (directly registering neuron activity) or optical (registering, for example, changes in oxygenation) means.

### Treatment of Tissue at Depth Specifically to Relieve Pain and Promote Healing

The methods and devices described are applicable, among other things, to treatments directed to the combined non-thermal photochemical effects (taking place in a physiological temperature range) induced by absorption of non-destructive narrow-band electromagnetic radiation and photothermal effects (32° C.-45° C.). Such treatments have been found in many studies to have a beneficial impact on the reduction of pain and the promotion of healing. These effects are preferably induced using narrow-band optical radiation, which can both produce the desired photochemical effects and elevate temperature in the target region.

### Pain Reduction

The embodiments described below can be used to reduce or relieve pain associated with the tissue to be treated. To effectively reduce or relieve pain by treating target tissue with EMR, several strategies can be used. Examples of such strategies are: vasodilation; LILT modulation of transmission of pain signals through neurons; reducing the inflammation at an injury site; and stimulation of production of endogenous hormones suppressing pain (e.g., endorphins).

Vasodilation is the variation of blood vessel permeability, facilitating passage of cellular blood components and blood plasma into the interstitial space. This process can have a direct effect on inflammation affecting pain.

The theory that the transmission of pain signals through neurons can be modulated using LILT is based on the concept that a biochemical process controls neuron impedance, and that the neuron impedance can be altered using LILT. The change of neuron impedance can affect the process of pain signal transmission from a peripheral source to a regional plexus and, subsequently, to the brain. The interruption of transmission of pain signals can occur at various locations, e.g., Rolando's substantia gelatinosa.

Inflammation at an injury site can be reduced through inhibition of cytokine expression. As an example, the COX-2 expression, which in turn regulates the production of prostaglandins E 2 and I 2 that mediate inflammation, can be down regulated.

The endogenous hormones suppressing pain (e.g., endorphins) can be stimulated to increase the production and reduce pain. This can occur through several intermediate pathways, either as a result of direct exposure of endorphin-producing centers to light or as a mediated response to peripheral exposure.

Pain reduction and healing can be initiated a number of ways, including by applying narrow-band optical radiation. To more effectively address any unwanted or excessive heating of the tissue being treated, several approaches can be used in addition to the cooling discussed above.

For example, pulsed (as opposed to Continuous Wave) irradiation can also be used to limit the temperature rise and maintain a safe treatment regime. Pulsewidths and intervals between pulses can be selected to allow sufficient thermal relaxation between two consecutive pulses. For treatment of human tissue, pulse durations preferably are between 100 msec and 2 sec, and the intervals between pulses preferably are between 20 ms and 2 s. The duty cycle of the train of pulses can vary between 10 and 100 percent.

The pulse sequence can also be optimized to provide maximal efficacy of treatment. For example, a pulse sequence can begin with a single hyperthermic pulse, creating an area of elevated temperature, followed by a train of lower-intensity, pain-mediating pulses. Similarly, pulses can be synchronized with biological cycles like heartbeat.

An additional consideration in optimizing a treatment device for relieving or reducing pain is the wavelengths of light that are to be used. At least two aspects should be considered. First, the wavelength of light should be chosen to optimize the depth of treatment as discussed herein. The optimal wavelengths for this purpose are discussed below. Second, because the wavelengths that provide for optimal penetration of tissue can not coincide with the wavelengths that are optimal for chromophore absorption, a second wavelength can be necessary for some treatments. The optimal wavelengths for chromophore absorption are discussed in the '705 patent application, referred to above.

In another aspect of the invention, the tissue is treated using radiation at different intensities. Preferably, an initial treatment is performed at a relatively higher intensity, with subsequent treatments being performed at lower intensities. Clinical tests have revealed that the human body compensates for chronic and other types of pain by altering the sensitivity of the body to the sensation of pain. Thus, for example, when a damaged muscle or other tissue causes pain for an extended period, the body effectively becomes desensitized to it. This change in the level of sensation is more than an alteration of the perception of pain. The alteration appears to manifest itself physically as well. For example, certain processes associated with healing and pain can not be effectively modulated or initiated using LILT at relatively lower intensities, because these processes become less susceptible to stimulation with EMR, and respond only if a much high intensity is used, at least initially.

Tests have shown that damaged muscle tissue is less responsive to such EMR therapy during initial treatments using EMR that are performed at relatively lower levels. The initial treatment at a given intensity of EMR can be ineffective in some patients, or the long term effect of the treatment can not be satisfactory, even if an initial reduction in pain were found. These tests have demonstrated that it is preferable to initially treat tissue, such as damages muscle tissue or joints, at a higher intensity. If the initial treatment is performed at an intensity that is too low, the body can not respond adequately or at all to treatment with EMR and can continue to be ineffective in subsequent treatments.

Instead, it is preferable to perform the initial treatment using EMR at a level of intensity above a threshold that is sufficient to alter the response of the tissue being treated. If the initial treatment or treatments are performed above such a threshold, subsequent treatments become effective using much low intensities. In effect, treating initially with higher intensities causes a biological system that can have become desensitized to a tissue injury to increase the “gain” of the system to normal levels.

Although the exact threshold that must be surpassed in the initial treatment varies from subject to subject and is difficult to precisely quantify, tests have shown that the threshold intensity is typically met when the subject reports a sensation of deep heating within the tissue being treated, i.e., a sharp sensation of heat that does not damage the tissue or leave a lingering sensation of pain. In cases where a higher intensity was used initially until the subject reported such a deep-heating sensation, the tissue became responsive to treatment at much lower intensities in subsequent treatments. In cases where a higher intensity was not used initially or the subject did not report a deep-heating sensation, the subjects did not consistently respond to subsequent treatments. In some cases the treatments were effective, in other cases there was no perceived or measured effect or the results were inconclusive. Without being limited by theory, there are several theories as to why this effect has been observed. One such theory is that the EMR delivered in relatively high intensities acts as a form of prolotherapy, stimulating the natural healing responses.

In certain embodiments, initial treatments can be performed at relatively higher intensities (e.g., approximately 0.8 watts/cm²-1.6 watts/cm² higher) and levels of power (e.g., 40 watts-80 watts or higher), and subsequent treatments can be performed at relatively lower intensities (e.g., 0.4-0.8 W/cm²_) corresponding to lower levels of power (e.g., 20-40 W). Preferably, the intensity is not sufficient to damage the tissue being treated, such as burning the skin that is irradiated. The initial high-intensity treatment(s) can require more aggressive parallel cooling of skin surface than subsequent lower-intensity treatments. Although the embodiments are described with reference to the ranges above, the exact values can vary from subject to subject and application to application due to the myriad of variables that will affect the parameters, including, without limitation, tissue type, tissue density, tissue composition, tissue volume location, the presence of multiple tissue types within a volume of tissue, and blood flow within tissue.

In another embodiment, the EMR can be applied at an initial intensity and, if there is no response, the intensity can be increased until the subject being treated experiences a sensation of heating as described above. Once that intensity is found, the EMR can be applied for a duration of time. Preferably, the EMR is applied at an intensity that does not cause severe pain, but that pushes the subject's ability to tolerate the treatment without experiencing excessive discomfort.

In such a method, the person applying the EMR, such as a physician, will determine the highest intensity of EMR that can be safe tolerated by the subject, and will apply the EMR at that intensity for as long as the subject can tolerate it (or until the treatment is completed). If the subject is unable to tolerate the treatment, the physician can “titrate” the intensity of the radiation by reducing it to a lower value that will be applied for the duration of the treatment. In effect, the intensity of EMR that likely will be most effective for a given subject will be an intensity that the subject cannot endure comfortably for the entire duration of the treatment. In other words, the overall treatment duration likely will exceed the duration of time that the maximum intensity level of EMR can be applied without causing the subject pain or severe discomfort. Thus, a lower intensity (or intensities) can be required at some point(s) in the treatment.

In an exemplary embodiment, the initial input flux will be in the range of 0.1-0.6 watts/cm². If the subject does not report a sensation of heating or pain, the input flux can be increased on the order of two to three times to a value in the range of 0.6 to 1.8 watts/cm². (It should be noted that cooling likely will be required for any input flux above 1.5 watts/cm², because most people will experience pain at or around that intensity.) When the person applying the EMR determines the maximum intensity or input flux that the subject can tolerate without experiencing pain or severe discomfort or otherwise damaging the tissue, that maximum input flux will be applied for as long as the subject is able to tolerate it without experiencing severing discomfort, pain or damage to the tissue. At that point in time, assuming the overall treatment period is not completed, the input flux can be reduced by, for example, 10-20% for the duration of the treatment, or, if necessary, can be further reduced multiple times, if the subject can no longer tolerate even the reduced intensity of EMR.

Treatment periods will vary depending on several parameters, including, without limitation, the type of tissue being treated, the volume, the depth, and the responsiveness of the subject being treated. A typical treatment will last approximately, for example, 3.5-5 minutes. However, many different treatment times are possible, including much shorter times, such as, e.g., treatments on the order of seconds, to much longer treatment times, such as, e.g., on the order of one or more hours. To assist the process and eliminate some of the trial and error in determining the proper input flux to apply, the treatment parameters can be automatically or manually recorded, so that, for example, a system having processing power can automatically determine the treatment parameters, such as timing and input flux, for use during the treatment or during subsequent treatments.

Future treatments can be performed in a similar manner, i.e., with the input flux at a maximum value for as long as the subject can tolerate the treatment and then at a reduced value or values through the remainder of the treatment. As discussed above, it is expected (but not required) that the maximum input flux will be lower during the subsequent treatments due to the change in the “gain” of the subject's system in response to initial treatment.

Alternatively, an initial treatment or treatments can be performed by more powerful equipment in a professional setting while subsequent treatments can be performed using lower power equipment, for example, in the home using a consumer device available by prescription or for general sale. Furthermore, lower intensity treatments can be performed to control pain and/or promote healing between treatments using higher intensities that can be performed, e.g., by a doctor and/or in a professional setting. Such low intensity treatments could also be used to allow a subject to maintain a biological effect (e.g., those associated with reducing chronic pain and/or promoting healing) for a period time until a treatment using a higher intensity of EMR is required, e.g., when there is a resumption of or a marked increase in the level of pain. Such embodiments allow those experiencing incurable chronic pain to be treated in a manner that will significantly decrease the level of pain, which can then be maintained for a longer and potentially extended period of time by using lower intensity treatments in between the higher intensity treatments.

### Healing of Damaged Tissue Using LILT

The embodiments described herein can also be used to promote the healing of wounds and other damaged tissue. As discussed above, recent studies have begun to illustrate that both fluence (i.e., dose) and fluence rate (i.e., irradiance) have an effect on healing. The bi-phasic effect of light and other EMR on cells and the healing process is also now the subject of study. To effectively promote healing by treating target tissue with EMR, several strategies can be used. Examples of such strategies are: biostimulation of cellular respiratory processes such as ATP production or cytochrome c oxidase; stimulation of an inflammatory response; irradiation of soft tissue below the surface; irradiation of tissues associated with pain and/or shown to be damaged.

As discussed above, cellular respiratory processes are thought to play a role in wound healing, and the photobiostimulation of tissue in an affected area can result in improved healing. For example, cytochrome c oxidase is a respiratory chain enzyme residing within the cellular mitochondria, and is the terminal enzyme in the respiratory chain of eukaryotic cells. Cytochrome c oxidase mediates the transfer of electrons from cytochrome c to molecular oxygen. The involvement of cytochrome c is known to be central to the redox chemistry leading to generation of free energy that is then converted into an electrochemical potential across the inner membrane of the mitochondrion, and ultimately drives the production of adenosine triphosphate (ATP).

It has been further demonstrated that photobiostimulation can be used to enhance cellular proliferation to achieve therapeutic effects by stimulating the production of ATP molecules to help generate cAMP, which is a secondary messenger affecting a multitude of physiological processes such as signal transduction, gene expression, blood coagulation and muscle contraction. Also, it is believed that there is an additional healing benefit achieved by stimulating increased blood to the affected area.

Accordingly, experiments conducted in vitro have demonstrated that photobiostimulation has the potential of increasing the energy available for metabolic activity of cells, and have also demonstrated that an increase in ATP production by photobiostimulation can provide a means to increase cell proliferation and protein production. The clinical research in this area, however, remains inconclusive at this time.

Similarly, it has been postulated that photobiostimulation using LLLT and similar radiation treatments can result in a change in the cellular redox state, which in turn can play a role in maintaining cellular activities. There is research that suggests that stimulation of tissue with laser, optical or other radiation can result in the formation of small amounts of light-induced reactive oxygen species (ROS) and antioxidants, which change the cellular redox state and stimulate cell processes. (See, e.g., Lubart R. et al., “Low-Energy Laser Irradiation Promotes Cellular Redox Activity,” Photomedicine and Laser Surgery, Vol. 23, No. 1, 2005, pp. 3-9.) ROS and antioxidants can be generated in various cell structures, such as, without limitation, cell structures produced by the mitochondria and in plasma membranes. In such processes, EMR can be absorbed by a chromophore, such as an intracellular chromophore. The EMR is applied at an appropriate wavelength, intensity and energy dose based on physical characteristics of the chromophore (or the various types of chromophores, if several are involved). Typical endogenous chromophores include, but are not limited to, porphyrins, flavins, mitochondrial cytochromes, the plasma membrane NADPH oxidase system, flavorproteins, and cytochrome b. The chromophores act as photosensitizers, and absorb EMR, such as visible light, and transfer it to nearby oxygen molecules, thus producing the ROS and/or antioxidants. High amounts of the ROS can be lethal to a cell. Therefore, the localized production of ROS can be induced to extinguish all cell activity in the location. However, if present in lower concentrations, for example, below that required for cytotoxicity, ROS can have a range of positive effects on the cells and surrounding tissue, for example, the stimulation of cell growth and the differentiation of neurons. Further, by targeting chromophores that are unique to certain types of cells in a region of tissue, only those cells or predominately those types of cells can be extinguished, stimulated, etc. Similarly, by targeting certain tissues or the blood itself, ROS levels can be increased in the bloodstream to promote broader systemic benefits, for example, by being transported to other parts of the body or more deeply within the tissue being treated.

Another potential mechanism to effectively promote wound healing is stimulation of an inflammatory response. For example, tissue can be irradiated to cause a limited irritation to the blood cells and walls in the vessels of the dermis. This results in a low-grade inflammatory/growth response. Inflammatory mediators are released through the vessel walls that stimulate fibroblast activity and eventually lead to a “healing” effect.

The tissue within the vascular system can be irradiated to promote healing. For example, vascular tissue below the surface can be irradiated to promote the healing of venous ulcerations and other disorders that are generally presently treated using invasive surgical procedures. Thus, such treatments can eliminate the need for surgery in some cases.

Similarly, pain that is caused by damage to tissues in the joints, such as ligaments, tendons and cartilage, can be treated. Where pain is attributed to volumes containing such tissues, the tissue can be treated to promote healing, even where damage to the tissue can not be readily apparent.

Other embodiments can use technical means of temperature monitoring, e.g. contact or IR thermometers with subsequent feedback to the power control unit.

### Prevention of Scar Formation and Fibrosis Using LILT

The embodiments described herein can also be used to eliminate or at least reduce formation of scar tissue and fibrosis resulting from surgical procedures, wounds, traumas and other pathogenic factors.

Mechanism of action specifically relevant for preventing scar formation and fibrosis involves light-induced modification of cytokine secretion by specialized cells, such as neutrophils, macrophages, lymphocytes, fibroblasts, etc. The feasibility of modulating cytokine secretion with light has been demonstrated for a number of cytokines, including Interleukin-1 (IL-1), tumor necrosis factor-α(TNF-α), interferon-γ (INF-γ), interleykin-4 (IL-4), interleykin-8 (IL-8) and others.

Without being limited by theory, at least some medical research has demonstrated that the phases that occur after muscle injury (phases of degeneration, inflammation, regeneration, and fibrosis) occur through a fluid continuum rather than at discrete times. The degenerative phase occurs during the first 48 hours post-injury. The inflammatory phase begins 48-96 hours after muscle injury. The regenerative phase begins approximately 1 week post-injury, peaks over the subsequent week, and then steadily declines. It has been postulated that, if the regenerative phase were allowed to proceed uninterrupted, the muscle would most likely heal without scarring. However, this phase ends prematurely due to the simultaneous production of fibrous tissue, which can be excessive in some cases. The fibrotic phase thus ultimately determines the extent of muscle healing. (See Fu F. H., Weiss K. R. and Zelle B. A., “The accelerated Rehabilitation of the Injured Athlete”, XIV International Congress on Sports Rehabilitation and Traumatology, 2005.)

Some embodiments according to the present invention allow the control of the healing process in muscle tissue by modulating the production of fibroblasts that both contribute to the formation of scar tissue and limit to healing of the muscle tissue. Various treatment regimes directed at prevention of scar formation and modulation of the fibrotic phase of the healing process can be performed using embodiments of the present invention. For example, in one embodiment, electromagnetic radiation having a wavelength of 830_nm can be applied to damaged skin or muscle tissue at a power density of 20 mW/cm² to control the formation of scar tissue by controlling to production of fibroblasts. Many other embodiments are possible.

In alternate embodiments, radiation can be used to modulate other processes associated with healing of muscle and other tissues as well as the formation of scars in muscle and other tissues, such as, without limitation, the rate of reaction of the immune system. Furthermore, photobiostimulation procedures can be performed either simultaneously or immediately after a surgical procedure by a medical professional. Additionally or alternatively, as with many of the potential treatments and applications including without limitation the prevention of scarring and the treatment of scars, other procedures can be performed and, depending on the steps involved, can be performed by persons of various skill levels including by a doctor, otherwise in a professional setting, and by a person using a device designed for home use, either by prescription or generally available for sale to the public.

### Sports Performance and Trauma Prevention

Additionally, embodiments according to the invention, can be used to enhance sports performance and prevent trauma to tissue. For example, low level EMR therapy can be used to provide deep heating of muscle tissue, which will have beneficial effects such as, for example, increasing the circulation of blood and increasing oxygenation of the tissue. Such treatments can be used to improve performance, prevent initial trauma, and/or to prevent re-injury to previously damaged muscle or other tissue that has healed (or substantially healed).

Similarly, alternative embodiments could increase free O₂, for example, by stimulating the mitochondria in cells. By applying EMR to an area or volume of tissue, the oxygen in the blood can be drawn into the tissue due to the increased respiration of the stimulated cells, potentially causing higher levels of O₂ in the tissue. The effect would be similar to that achieved by the use of hyperbaric chambers by athletes to expedite healing, prevent injury, or improve performance, e.g., of muscle tissue. An optical treatment can require cooling of the tissue surface to allow the use of higher input fluxes. For example, a system with cooling could be used or a contact gel.

Other embodiments are possible, such as treatments with higher intensities of EMR than those typically used for low level light therapies.

### Potential Treatments and Treatment Parameters

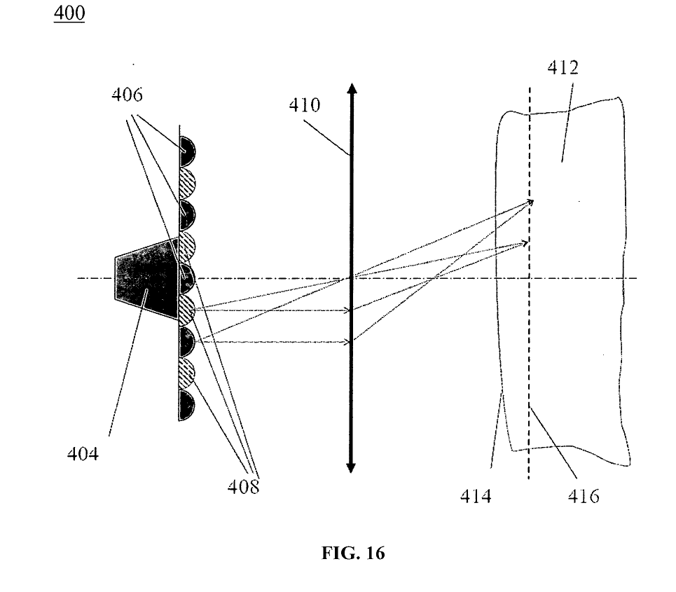

Many alternative embodiments are possible, including various devices and methods. For example, referring to FIG. 16, a device for treating a predefined volume of tissue can have a light source assembly including a source for generating EMR of multiple wavelengths in the range between 350 and 1900 nm. Different wavelengths can be generated either simultaneously or sequentially. Such a device can include a radiation source assembly 400 that includes a controller 404, first wavelength sources 406, second wavelength sources 408 and an optical imaging system 410. The controller 404 controls the power from the power source (not shown) as well as the timing and sequencing of radiation that is emitted from the wavelength sources 406 and 408. First wavelength sources 406 are LEDs that emit radiation at a wavelength of 350 nm. Second wavelength sources 408 are LEDs that emit radiation at a wavelength of 1900 nm. The radiation is transmitted through optical imaging system 410, in this case a convex lens. Alternatively, the optical imaging system can be a system of lenses or other mechanisms.

During operation, optical imaging system 410 images the radiation emitted from sources 406 and 410 onto a tissue treatment area 412 at a depth below a surface 414 of the tissue being treated. An image plane 416 at which the radiation is focused can be located at various depths depending on the design and application and can also be located at the surface.

Various embodiments of the invention can use different combinations of wavelengths, both in specific wavelengths used and in the number of different wavelengths used, e.g., two, three or more different wavelengths. Some embodiments can use one or several separate narrow bands (FWHM up to 50 nm) in combination with one or several broad bands (FWHM>50 nm). The purpose of such combinations can also vary depending on the application and/or treatment. Additionally, various treatments can be combined where, for example, the treatments are found to by synergistic and/or when the efficacy of the treatments is not reduced when combined.

Although the imaging system is referred to as an optical imaging system, unless otherwise specified, the term optical and its derivatives (such as optically) as used herein is meant to additionally encompass electromagnetic radiation of wavelengths outside the spectrum of visible light. Further, although many embodiments are described in the context of using visible light, the scope of the invention encompasses EMR generally, as well as other forms of radiant energy, such as acoustic waves, ultrasound, etc.

Depth of light penetration is determined by tissue types and wavelength. Wavelengths of 632 nm (He—Ne), 670 nm (InGaAlP), 810 nm/830 nm (GaAlAs), 850 nm/904 nm, LED (e.g., 660 nm) have been used as light source with positive results. The most frequently used wavelength is 810 nm/830 nm due to its availability, effect and presumed good penetration depth. Wavelengths of 632 nm (He—Ne) have lesser penetration capabilities.

Additionally, to treat tissue at depth, wavelengths 380-610 nm or 1400-10000 nm can be used for superficial target treatment or 610-1400 nm for deep target treatment. More preferably, the following wavelengths of LILT can be used: 400 to 430 nm, 480 to 520 nm, 570 to 690 nm, 750 to 780 nm, 800 to 840 nm, 880 to 920 nm, 950 to 1100 nm. Table III below lists preferred parameters of irradiation. For treatment of muscle and joint paint (such as temporomandibular joint (TMJ) pain), the following parameters can be used: wavelength of 800 to 850 nm, input flux between 100 and 1000 (preferably between 200 and 600) mW/cm², pulsewidth between 0.5 and 2 sec. (preferably ˜1 sec.), duty cycle 10 to 90% (preferably ˜75%), treatment time between 1 and 20 min. (preferably between 1 and 5 min.)

In other embodiments, several narrow bands can be used to target different chromophores for inducing different pathways of photobiomodulation, or to induce photobiostimulation in different tissue volumes, due to difference in penetration depth. Alternatively, broad or narrow bands can be used to induce hyperthermia in tissue at desired tissue volumes and thus enhance biostimulation. For example, embodiments can utilize two narrow bands (WFHM between 1 and 40 nm) with maxima located in the spectral regions of 390 to 500 nm and 610 to 850 nm, respectively. Preferably, but not essentially, the maxima are in the ranges 405 to 450 nm and 800 to 830 nm, respectively.

In still other embodiments, photobiostimulating effects can be enhanced by elevating concentration or increasing sensitivity of primary endogenous chromophores. This can be achieved, for example, through topical or systemic application, prior to light treatment, of biological precursors of the chromophores. The precursors can be metabolized or otherwise processed by the body, resulting in the desired increase of the chromophore concentration. Alternatively, one can administer, prior to EMR treatment, a substance that possesses an affinity for the desired chromophores and, upon binding to molecules of the chromophores, changes their configuration so as to increase their sensitivity to light treatment. For example, an exemplary embodiment can utilize compounds of the vitamin B family, which are known to be biological precursors of molecules and substances that are relevant to producing the desired biostimulative effect, such as, for example, riboflavins, and chromophores relevant to treatments using radiation having a wavelength of 400 to 500 nm.

To irradiate tissue volumes at various depths, the following parameters outlined in Table II are considered preferable.

TABLE II
Preferred irradiation parameters for pain reduction and wound healing.

| Target | | | | |
|---|---|---|---|---|
| depth, | Pulse width | Repetition Rate | Input Flux | Irradiation |
| mm | (msec) | (Hz) | (mW/cm²) | Duration (sec) |
| 0.01–1 | 100–1000 | 0.1–10 | 50–90 | 20–300 |
| 0.2–2 | 100–1000 | 0.1–10 | 90–180 | 20–300 |
| 0.5–3 | 100–1000 | 0.1–10 | 180–270 | 20–300 |
| 1–4 | 100–1000 | 0.1–10 | 270–360 | 20–300 |
| 2–5 | 100–1000 | 0.1–10 | 360–530 | 20–300 |
| 3–6 | 100–1000 | 0.1–10 | 530–710 | 20–300 |
| 4–7 | 100–1000 | 0.1–10 | 710–890 | 20–300 |
| 5–8 | 100–1000 | 0.1–10 | 890–1070 | 20–300 |
| 7–9 | 100–1000 | 0.1–10 | 1070–1240 | 20–300 |
| 8–11 | 100–1000 | 0.1–10 | 1240–1420 | 20–300 |
| >10 | 100–1000 | 0.1–10 | 1420–1600 | 20–300 |
| | | | (or more) | |

Parameters of Table II have been computed for the case when the source of narrow-band light is also used to elevate the temperature in the tissue. However, other configurations are possible, including the use of other bands of EMR, such as near infrared, to elevate the temperature in the tissue.

The following is a non-exclusive list of conditions, which can be treated using the method of the present invention:
1. LBP/sciatica
2. Neck pain
3. Whiplash
4. Facet syndrome
5. Myofascial pain/trigger points
6. Interstitial cystitis
7. DJD of hands, knee, ankle, hip, feet (notice of accelerated nail growth with Rx of distal finger.)
8. CTS
9. Epicondylitis lateral & medial
10. Radiculitis
11. Plantar fasciitis
12. Biceps tendonitis
13. Patellar tendonitis
14. Hamstring tear
15. Ankle sprain
16. Medial collateral ligament strain
17. Trochanteric bursitis
18. Piriformis syndrome
19. AC joint arthroscopy/sprain
20. s/p ACL repair
21. Shin splint/posterior tibialis tendonitis
22. Rotator cuff tendonitis
23. Hip flexor strain
24. Fibromyalgia
25. Intercostal neuritis
26. Sacroilleitis
27. Edema associated with soft tissue/joint trauma
28. TMJ pain
29. Scar remodeling associated with surgical incisions
30. Metatarsalgia
31. Morton's neuroma
32. Ulnar Neuritis
33. DeQuervain's Tenosynovitis
34. Wrist pina-unspecified
35. Thoracic Outlet Syndrome
36. RSD reflex sympathetic dystrophy
37. Muscle strain/spasm
38. Tendinopathy
39. Wound Healing

It will be appreciated that many alternate embodiments and variations in the methods and devices that have been described are possible. For example, many additional applications to various treatment and treatment parameters beyond those described here are possible, and the disclosed treatment parameters can be varied to suit the desired treatment.

For example, the synergetic effect of EMR and oral or topical compounds can be used. These compounds can be any pain relief drugs, foods, herbs, lotions or it can be compound with pain relief effects induced by light. Light or other EMR can enhance or generate the reduction in pain relief due to either photochemical or photothermal effects. Light can enhance penetration of topical pain relief compound or promote delivery of a systemically administered compound into treatment area by increasing local microcirculation.

While several embodiments of the invention have been described and illustrated herein, those of ordinary skill in the art will readily envision a variety of other means and structures for performing the functions and/or obtaining the results and/or advantages described herein, and each of such variations or modifications are within the scope of the present invention.

For example, those skilled in the art will appreciate that while embodiments have been described in the context of EMR treatment systems, many other embodiments are possible. For example, devices other than treatment heads are possible. For example, where applications require longer treatment pulses or longer treatment times to treat tissue, devices that are not required to be held during operation would be advantageous. Thus, a device intended to treat one area of tissue for an extended period could be configured in the form of a pressure cuff or a stationary applicator pad that could be laid, taped, clipped, strapped, etc. to the person being treated.

More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. The present invention is directed to each individual feature, system, material and/or method described herein. In addition, any combination of two or more such features, systems, materials and/or methods, if such features, systems, materials and/or methods are not mutually inconsistent, is included within the scope of the present invention.

As used herein, EMR includes the range of wavelengths approximately between 200 nm and 10 mm. Optical radiation, i.e., EMR in the spectrum having wavelengths in the range between approximately 200 nm and 100 μm, is preferably employed in the embodiments described above, but, also as discussed above, many other wavelengths of energy can be used alone or in combination. The term “narrow-band” refers to the electromagnetic radiation spectrum, having a single peak or multiple peaks with FWHM (full width at half maximum) of each peak typically not exceeding 10% of the central wavelength of the respective peak. The actual spectrum can also include broad-band components, either providing additional treatment benefits or having no effect on treatment. Additionally, the term optical (when used in a term other than term “optical radiation”) applies to the entire EMR spectrum. For example, as used herein, the term “optical path” is a path suitable for EMR radiation other than “optical radiation.”

TABLE III
Typical parameters of treatment at depth of exemplary tissues:

| | | | | | Treatment parameters with precooling | | | | | Treatment parameters | | |
|---|---|---|---|---|---|---|---|---|---|---|---|---|
| | Depth | | | | for preferable wavelength range | | | | | without precooling for | | |
| | of peak | | | | Cooling | | | | | preferable wavelength range | | |
| | temper- | Wavelength range, μm | | | temper- | | | | | Cooling | Minimum | Input |
| | ature, | | | Most | ature, | Precooling | Time of | Fluence | | temper- | time of | Flux, |
| Organ | mm | Maximum | Preferable | Preferable | ° C. | time, s | treatment, s | J/cm² | W/cm² | ature, ° C. | treatment, s | W/cm² |
| Reticular | 1–3 | 0.5–1.85 | 0.5–1.0 | 0.5–0.7 | 5–25 | 0–30 | 60–6000 | 6–600 | 0.1–10 | 5–25 | 60–180 | 0.1–5 |
| dermis | | | & | & | | | | | | | | |
| | | | 1.5–1.8 | 1.6–1.8 | | | | | | | | |
| Hypo- | 2–5 | 0.6–1.35 | 0.6–1.1 | 0.6–1.0 | 5–25 | 0–30 | 120–800 | 12–2400 | 0.1–3 | 5–25 | 120–800 | 0.1–1.5 |
| dermis | | & | & | & | | | | | | | | |
| | | 1.6–1.8 | 1.65–1.8 | 1.7–1.75 | | | | | | | | |
| Muscle | 5–15 | 0.8–1.4 | 0.8–1.1 | 0.8–1.1 | 5–25 | 0–110 | 150–900 | 15–1800 | 0.1–2 | 5–25 | 150–900 | 0.1–1.8 |
| and | | & | & | | | | | | | | | |
| joint | | 1.6–1.7 | 1.65–1.8 | | | | | | | | | |
| | 10–20 | 0.8–1.3 | 1.1–1.25 | 1.15–1.23 | | 0–450 | 150–1200 | 15–1800 | 0.1–1.5 | 5–25 | 150–1200 | 0.1–1.3 |
| | 20–50 | 0.8–1.3 | 1.05–1.25 | 1.05–1.15 | | 0–900 | 150–1500 | 15–1800 | 0.1–1.2 | 5–25 | 150–1500 | 0.1–1.0 |

What is claimed is:

1. A device for treating a volume of tissue, comprising:
a source of EMR configured to transmit EMR to a tissue surface;
a controller electrically connected to said EMR source and configured to provide at least one control signal to said EMR source;
wherein said EMR source is configured to emit at least a first level of flux and a second level of flux in response to said controller, said first and second levels of flux corresponding to first and second penetration depths below the surface of the tissue.

2. The device of claim 1, wherein said controller includes a modulator in electrical communication with said EMR source to control said first and second levels of flux.

3. The device of claim 1, further including a cooling surface for contacting said tissue surface, said cooling surface configured to cool said tissue when in contact with said tissue surface during operation of said device.

4. The device of claim 1, further including a window configured to pass EMR.

5. The device of claim 4, wherein said window further includes a cooling surface for contacting said tissue surface, said cooling surface configured to cool said tissue when in contact with said tissue surface during operation of said device.

6. The device of claim 4, wherein said window has a radiation-passing area greater than approximately 49 cm².

7. The device of claim 4, wherein said window is configured to provide a variable radiation-passing area.

8. The device of claim 1, further comprising an aperture configured to pass radiation to said tissue.

9. The device of claim 8, wherein said aperture has an opening with a diameter greater than approximately 7 cm.

10. The device of claim 8, wherein said aperture is configured to have a variable size.

11. The device of claim 1, wherein said device is a handheld device.

12. The device of claim 1, wherein said device is a consumer product.

13. The device of claim 1, further comprising a feedback sensor configured to provide a feedback signal during operation;
wherein said controller is electrically connected to said feedback sensor mechanism and configured to issue said control signals based on said information obtained from said feedback sensor.

14. The device of claim 13, wherein said feedback sensor is a temperature sensor.

15. The device of claim 14, wherein said temperature sensor is configured to measure the temperature of said tissue being treated during operation.

16. The device of claim 13, wherein said feedback sensor is an optical Doppler sensor configured to measure the flow of blood within said tissue being treated.

17. The device of claim 1, wherein said EMR source is configured to provide an input flux between approximately 0.1 and 10 watts/cm².

18. The device of claim 1, wherein the device is configured to have a total system power greater than 40 watts.

19. The device of claim 1, wherein the device is configured to have a total system power greater than 80 watts.

20. The device of claim 1, wherein said source is configured to provide a minimally effective dose of EMR to tissue depths up to approximately 50 mm.

21. The device of claim 1, wherein said source is configured to provide a minimally effective dose of EMR to tissue depths up to approximately 20 mm.

22. The device of claim 1, wherein said source is configured to provide a minimally effective dose of EMR to tissue depths up to approximately 10 mm.

23. The device of claim 1, wherein said controller includes a memory device and a processor.

24. The device of claim 23, further comprising input sensors, wherein said controller derives treatment parameters using input data from said input sensors.

25. The device of claim 23, further comprising at least one feedback sensor in electrical communication with said controller, and wherein said controller is configured to compute at least one treatment parameter based on said sensor data.

26. The device of claim 1, wherein said controller includes a lookup table containing information regarding treatment parameters.

27. The device of claim 1, wherein said controller is configured to modulate said irradiance of EMR emitted from said source using intermittent pulses.

28. The device of claim 1, wherein said source further includes optical elements configured to provide an adjustable area of EMR that is incident on a surface of said tissue.

29. The device of claim 1 wherein said EMR source is configured to emit a third level of flux in response to said at least one control signal, said third level of flux corresponding to a third depth below the surface of the tissue.

30. A device for treating tissue, comprising:
a source for generating EMR;
an optical window for contacting a surface of said tissue to be treated and for transmitting EMR from said source to said tissue;
a cooling system in thermal communication with said optical window, said cooling system configured to remove heat from said optical window; and
a modulator electrically connected to said EMR source for varying a radiant flux emitted by said EMR source from a first value corresponding to a first tissue depth to a second value corresponding to a second tissue depth.

31. The device of claim 30, wherein said optical window is composed of sapphire.

32. The device of claim 30, wherein said device is a handheld device.

33. The device of claim 30, wherein said device is a consumer product.

34. The device of claim 30, further comprising a feedback sensor in electrical communication with said modulator;
wherein said modulator is configured to receive a feedback signal during operation and vary the flux emitted by said EMR source in response thereto.

35. The device of claim 34, wherein said feedback sensor is a temperature sensor.

36. The device of claim 35, wherein said temperature sensor is configured to measure the temperature of said tissue being treated.

37. The device of claim 35, wherein said feedback sensor is an optical Doppler sensor configured to measure the flow of blood within said tissue being treated.

38. The device of claim 30, wherein said source is configured to provide an input flux between approximately 0.1 and 10 watts/cm².

39. The device of claim 30, wherein said source is configured to irradiate tissue above a minimally effective threshold of irradiation at tissue depths selected from the group of ranges consisting of between approximately 0 and 50 mm, between approximately 0 and 20 mm, and between approximately 0 and 10 mm.

40. The device of claim 30, wherein said modulator includes a memory device and a processor.

41. The device of claim 40, further comprising at least one sensor in electrical communication with said modulator, wherein said modulator is configured to computes treatment parameters using signals from each of said at least one sensor.

42. The device of claim 30, wherein said modulator includes a lookup table containing information regarding treatment parameters.

43. The device of claim 30, wherein said modulator is configured to modulate said irradiance of EMR emitted from said source using intermittent pulses.

44. The device of claim 30, wherein said optical window comprises an area greater than approximately 49 cm².

45. The device of claim 30, wherein said source further includes optical elements configured to provide an adjustable area of EMR incident on a surface of said tissue.

46. The device of claim 30, wherein said modulator is electrically connected to said EMR source and is configured to vary said radiant flux emitted by said EMR source to a third value corresponding to a third tissue depth.

47. The device of claim 30, wherein said modulator is configured to vary said radiant flux within a continuous range.

48. The device of claim 30, wherein said modulator is configured to vary said radiant flux to a set of discrete values.

49. A device for transmitting light into tissue to treat damaged tissue or reduce pain, comprising:
a housing having an EMR source and an aperture for allowing EMR generated by said source to pass through said housing to said tissue;
wherein said source is configured to generate a flux of EMR passing through said aperture that is greater than or equal to approximately 0.1 W/cm².

50. The device of claim 49, wherein said aperture has a diameter in the range of about 1 cm to about 15 cm.

51. The device of claim 49, wherein said aperture has a diameter of at least about 7 cm.

52. The device of claim 49, wherein said device is configured to produce a beam of EMR having a cross-sectional area in the range of about 10 cm² to about 100 cm².

53. The device of claim 49, wherein said device is configured to produce a beam of EMR having a cross-sectional area of at least approximately 49 cm².

54. The device of claim 49, wherein said device is configured to produce a beam of EMR having a diameter of at least about 7 cm.

55. The device of claim 49, wherein said aperture is adjustable.

56. The device of claim 55, wherein said aperture is adjustable from a first area configured to produce a first level of flux to a second area configured to produce a second level of flux.

57. A device for transmitting light into tissue, comprising:
a housing having a window;
an EMR source mounted within said housing;
a set of optical elements mounted within said housing and forming an optical path extending between said EMR source and said window;
wherein said optical elements are adjustable to alter a spot size of EMR emitted from said window to the surface of the tissue to alter the input flux at the surface of the tissue, and
wherein the flux of said EMR emitted through said window is greater than or equal to about 0.1 W/cm².

58. A device for treating tissue at a predetermined depth below the surface of the tissue comprising:
a housing having a window;
an EMR source mounted within said housing, wherein said optical window allows EMR to pass through said housing to said tissue surface;
wherein said EMR source provides a level of flux corresponding to said predetermined depth and provides a power density of greater than or equal to about 0.1 watts/cm².

59. A method for irradiating tissue at depth, comprising:
selecting a first input flux corresponding to a first tissue depth; and
irradiating the tissue at said first depth using said first input flux.

60. The method of claim 59, wherein the step of irradiating further includes irradiating at a level that is above a minimum threshold of irradiance required to provide at least a minimally effective dose of EMR.

61. The method of claim 59, wherein the step of irradiating further includes irradiating at a level that is below a maximum threshold of irradiance required to provide at least a minimally effective dose of EMR.

62. The method of claim 59, wherein the step of irradiating further includes irradiating at a level that is above a minimum threshold of irradiance required to provide at least an effective dose of EMR and below a maximum threshold of irradiance required to provide at least an effective dose of EMR.

63. A method for treating a volume of tissue, comprising;
irradiating a surface of said tissue with EMR having a first power density; and
irradiating said surface with EMR having a second power density, wherein said first and second power densities correspond to a location of said volume of tissue to be treated.

64. The method of claim 63 further, comprising modulating between said first and second power densities according to a time varying function.

65. The method of claim 64, wherein said function is a continuous curve.

66. The method of claim 63, further comprising modulating between said first and second power densities by irradiating tissue at a set of discrete interim power densities.

67. The method of claim 63, further comprising modulating between said first and second power densities such that an applied power density remains above a minimum threshold of power densities that provide an effective dose of EMR to tissue at depth.

68. The method of claim 63, further comprising modulating between said first and second power densities such that an applied power density remains below a minimum threshold of power densities that provide an effective dose of EMR to tissue at depth.
